(19) United States
(12) Patent Application Publication
(10) Pub. No.: US 2025/0312677 A1
(43) Pub. Date: Oct. 9, 2025

(54) USER INTERFACES FOR MANAGING A WORKOUT SESSION

(71) Applicant: Apple Inc., Cupertino, CA (US)

(72) Inventors: Margaret A. BIGNELL, San Francisco, CA (US); Craig D. BOLTON, South Coogee (AU); Abhiraj S. PANDE, Santa Clara, CA (US)

(21) Appl. No.: 19/030,796

(22) Filed: Jan. 17, 2025

Related U.S. Application Data
(60) Provisional application No. 63/631,396, filed on Apr. 8, 2024.
(60) Provisional application No. 63/649,298, filed on May 17, 2024.
(60) Provisional application No. 63/657,066, filed on Jun. 6, 2024.

(51) Int. Cl. A63B 71/06; A63B 24/00

(52) U.S. Cl. CPC A63B 71/0622; A63B 24/0062; A63B 2071/0675; A63B 2220/17; A63B 2220/20; A63B 2220/72; A63B 2244/20

(57) ABSTRACT

The present disclosure generally relates to managing workout sessions. A computer system determines whether to initiate a workout session or display a notification that enables one or more goals of the workout session to be updated based on a selected distance associated with the workout session.

### CROSS-REFERENCE TO RELATED APPLICATIONS

This application claims priority to U.S. Provisional Application Ser. No. 63/631,396, entitled “USER INTERFACES FOR MANAGING A WORKOUT SESSION,” filed Apr. 8, 2024, U.S. Provisional Application Ser. No. 63/649,298 entitled “USER INTERFACES FOR MANAGING A WORKOUT SESSION,” filed May 17, 2024, and U.S. Provisional Application Ser. No. 63/657,066, entitled “USER INTERFACES FOR MANAGING A WORKOUT SESSION,” filed Jun. 6, 2024, the entire contents of each of which are hereby incorporated by reference.

### FIELD

The present disclosure relates generally to computer user interfaces, and more specifically to techniques for managing workout sessions.

### BACKGROUND

Electronic devices can be used to track workouts of a user and display information related to fitness activity of a user while the user performs the workout.

### BRIEF SUMMARY

Some techniques for managing workout sessions using electronic devices, however, are generally cumbersome and inefficient. For example, some existing techniques use a complex and time-consuming user interface, which may include multiple key presses or keystrokes. Some existing techniques limit an amount of customization that can be performed on workout sessions and/or require multiple user inputs to create a workout, start a workout, and/or transition between portions of a workout. Existing techniques require more time than necessary, wasting user time and device energy. This latter consideration is particularly important in battery-operated devices.

Accordingly, the present technique provides electronic devices with faster, more efficient methods and interfaces for managing workout sessions. Such methods and interfaces optionally complement or replace other methods for managing workout sessions. Such methods and interfaces reduce the cognitive burden on a user and produce a more efficient human-machine interface. Such methods also reduce a number of inputs needed to adjust goals of a workout session and/or provide information about reaching one or more goals of respective portions of a workout without requiring additional user input. For battery-operated computing devices, such methods and interfaces conserve power and increase the time between battery charges.

A method is described, in accordance with some embodiments. The method is performed at a computer system that is in communication with a display generation component and one or more input devices. The method comprises: prior to initiating a workout session, prompting a user of the computer system to input a distance associated with the workout session; after prompting the user of the computer system to input the distance associated with the workout session, detecting, via the one or more input devices, one or more user inputs selecting the distance associated with the workout session; and after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A non-transitory computer-readable storage medium is described, in accordance with some embodiments. The non-transitory computer-readable storage medium stores one or more programs configured to be executed by one or more processors of a computer system that is in communication with a display generation component and one or more input devices, the one or more programs including instructions for: prior to initiating a workout session, prompting a user of the computer system to input a distance associated with the workout session; after prompting the user of the computer system to input the distance associated with the workout session, detecting, via the one or more input devices, one or more user inputs selecting the distance associated with the workout session; and after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A transitory computer-readable storage medium is described, in accordance with some embodiments. The transitory computer-readable storage medium stores one or more programs configured to be executed by one or more processors of a computer system that is in communication with a display generation component and one or more input devices, the one or more programs including instructions for: prior to initiating a workout session, prompting a user of the computer system to input a distance associated with the workout session; after prompting the user of the computer system to input the distance associated with the workout session, detecting, via the one or more input devices, one or more user inputs selecting the distance associated with the workout session; and after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A computer system is described, in accordance with some embodiments. The computer system is configured to communicate with a display generation component and one or more input devices and comprises: one or more processors; and memory storing one or more programs configured to be executed by the one or more processors, the one or more programs including instructions for: prior to initiating a workout session, prompting a user of the computer system to input a distance associated with the workout session; after prompting the user of the computer system to input the distance associated with the workout session, detecting, via the one or more input devices, one or more user inputs selecting the distance associated with the workout session; and after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A computer system is described, in accordance with some embodiments. The computer system is configured to communicate with a display generation component and one or more input devices and comprises: means for, prior to initiating a workout session, prompting a user of the computer system to input a distance associated with the workout session; means for, after prompting the user of the computer system to input the distance associated with the workout session, detecting, via the one or more input devices, one or more user inputs selecting the distance associated with the workout session; and means for, after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A computer program product is described, in accordance with some embodiments. The computer program product comprises one or more programs configured to be executed by one or more processors of a computer system that is in communication with a display generation component and one or more input devices, the one or more programs including instructions for: prior to initiating a workout session, prompting a user of the computer system to input a distance associated with the workout session; after prompting the user of the computer system to input the distance associated with the workout session, detecting, via the one or more input devices, one or more user inputs selecting the distance associated with the workout session; and after receiving the one or more user inputs selecting the distance associated with the workout session: in accordance with a determination that the distance associated with the workout session satisfies a set of criteria, initiating the workout session; and in accordance with a determination that the distance associated with the workout session does not satisfy the set of criteria, displaying, via the display generation component, a notification without initiating the workout session, wherein the notification includes: a first selectable user interface object that, when selected via user input, initiates a process to update one or more goals of the workout session.

A method is described, in accordance with some embodiments. The method is performed at a computer system that is in communication with a display generation component and one or more input devices. The method comprises: while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

A non-transitory computer-readable storage medium is described, in accordance with some embodiments. The non-transitory computer-readable storage medium stores one or more programs configured to be executed by one or more processors of a computer system that is in communication with a display generation component and one or more input devices, the one or more programs including instructions for: while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

A transitory computer-readable storage medium is described, in accordance with some embodiments. The transitory computer-readable storage medium stores one or more programs configured to be executed by one or more processors of a computer system that is in communication with a display generation component and one or more input devices, the one or more programs including instructions for: while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

A computer system is described, in accordance with some embodiments. The computer system is configured to communicate with a display generation component and one or more input devices and comprises: one or more processors; and memory storing one or more programs configured to be executed by the one or more processors, the one or more programs including instructions for: while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

A computer system is described, in accordance with some embodiments. The computer system is configured to communicate with a display generation component and one or more input devices and comprises: means for, while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and means for, in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

A computer program product is described, in accordance with some embodiments. The computer program product comprises one or more programs configured to be executed by one or more processors of a computer system that is in communication with a display generation component and one or more input devices, the one or more programs including instructions for: while detecting, via the one or more input devices, activity information associated with an active workout session that includes a first interval corresponding to a first distance goal and a first duration and a second interval corresponding to a second distance goal and a second duration, receiving, via the one or more input devices, an indication that the first distance goal corresponding to the first interval of the active workout session has been reached; and in response to receiving the indication that the first distance goal corresponding to the first interval of the active workout session has been reached: in accordance with a determination that the first distance goal corresponding to the first interval of the active workout session has been reached within the first duration corresponding to the first interval of the active workout session, displaying, via the display generation component, an indication of an amount of time remaining in the first duration corresponding to the first interval of the active workout session without initiating the second interval of the active workout session, wherein the computer system is configured to cease updating the indication of the amount of time remaining in the first duration after the first duration ends; and in accordance with a determination that the first distance goal corresponding to the first interval of the active workout session has not been reached within the first duration corresponding to the first interval of the active workout session, initiating the second interval of the active workout session.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing workout sessions, thereby increasing the effectiveness, efficiency, and user satisfaction with such devices. Such methods and interfaces may complement or replace other methods for managing workout sessions.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6AB illustrate exemplary user interfaces for managing workout sessions, in accordance with some embodiments.

FIG. 7 is a flow diagram illustrating a method for updating one or more goals of workout sessions, in accordance with some embodiments.

FIG. 8 is a flow diagram illustrating a method for displaying information about respective intervals of workout sessions, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for managing workout sessions. For instance, there is a need for electronic devices that suggest updates to and/or automatically update one or more goals of a workout session based on a distance selected for the workout session. In addition, there is a need for electronic devices that initiate a next interval of a workout session and/or provide information about an amount of remaining time in a current interval of the workout session based on when a target distance is reached. Such techniques can reduce the cognitive burden on a user who performs workout sessions, thereby enhancing productivity. Further, such techniques can reduce processor and battery power otherwise wasted on redundant user inputs.

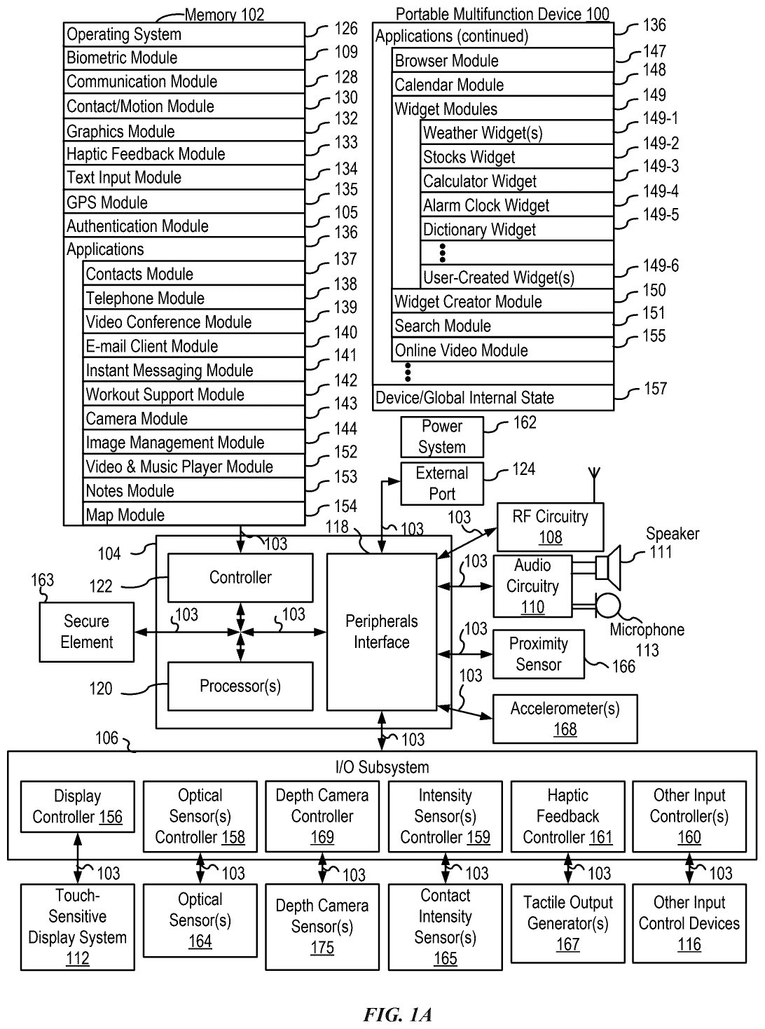

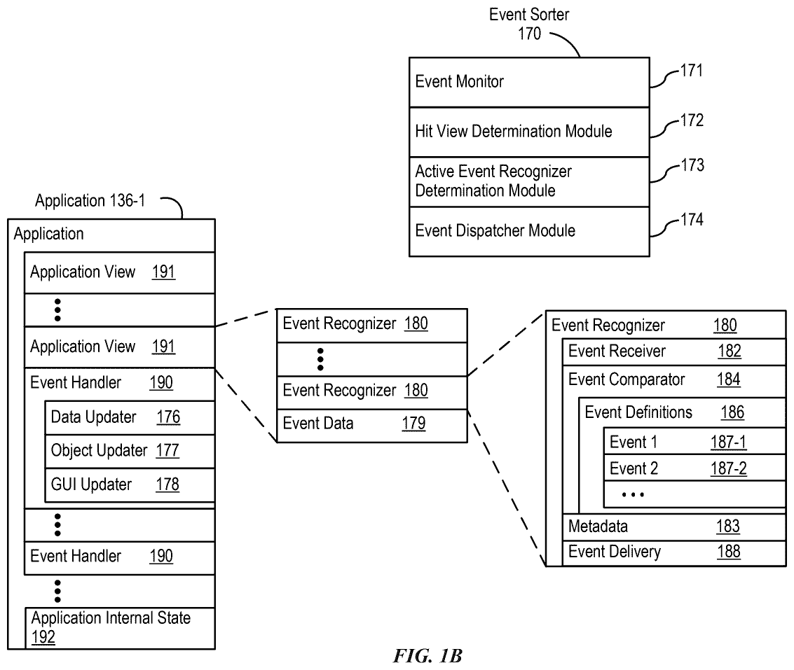

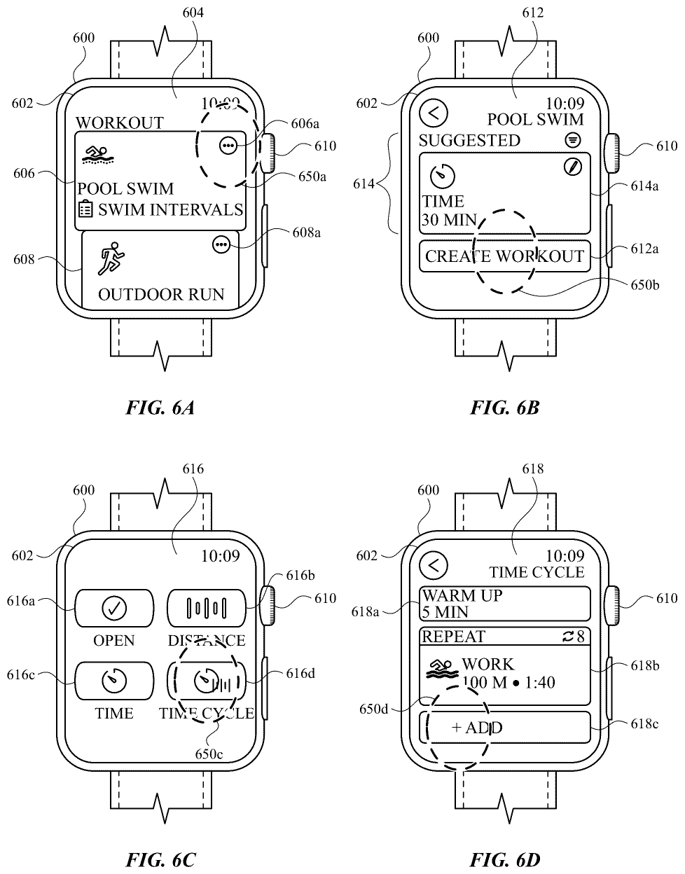

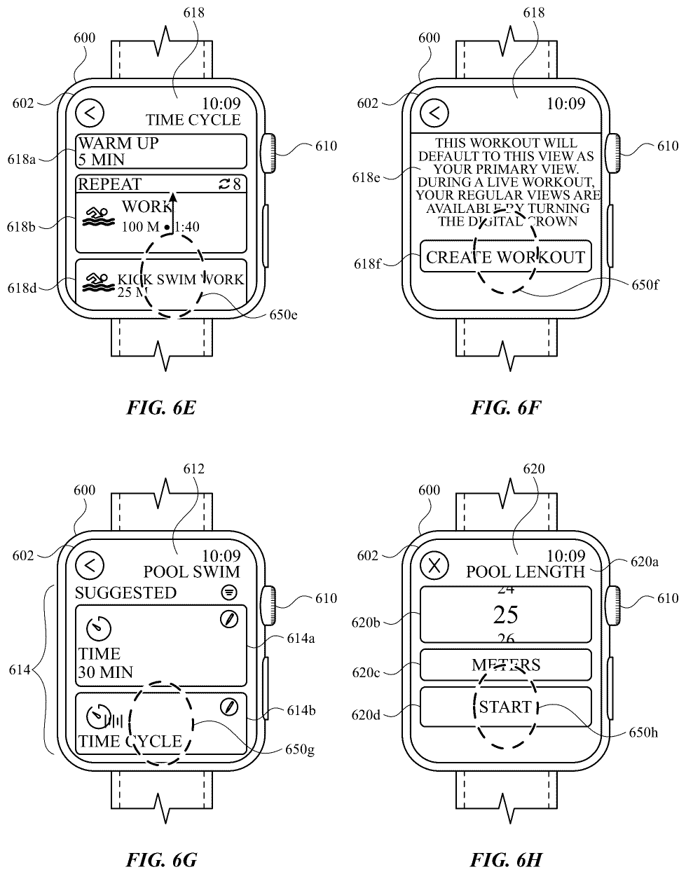

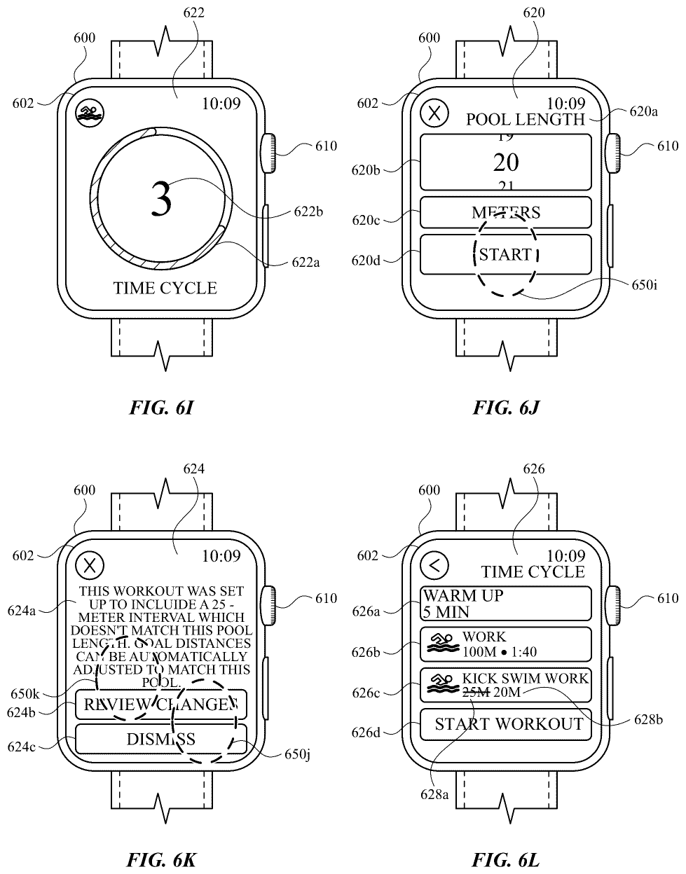

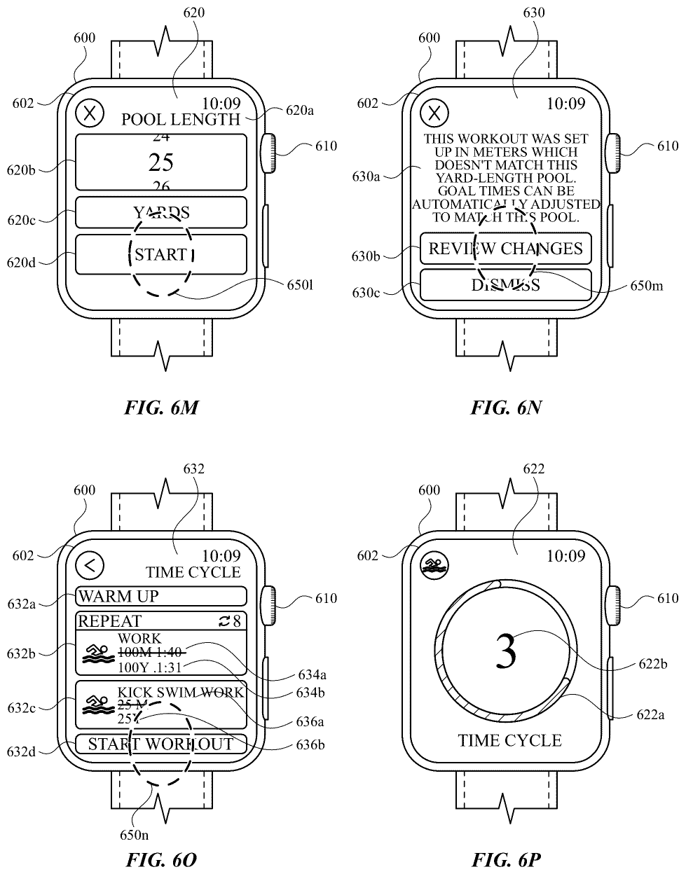

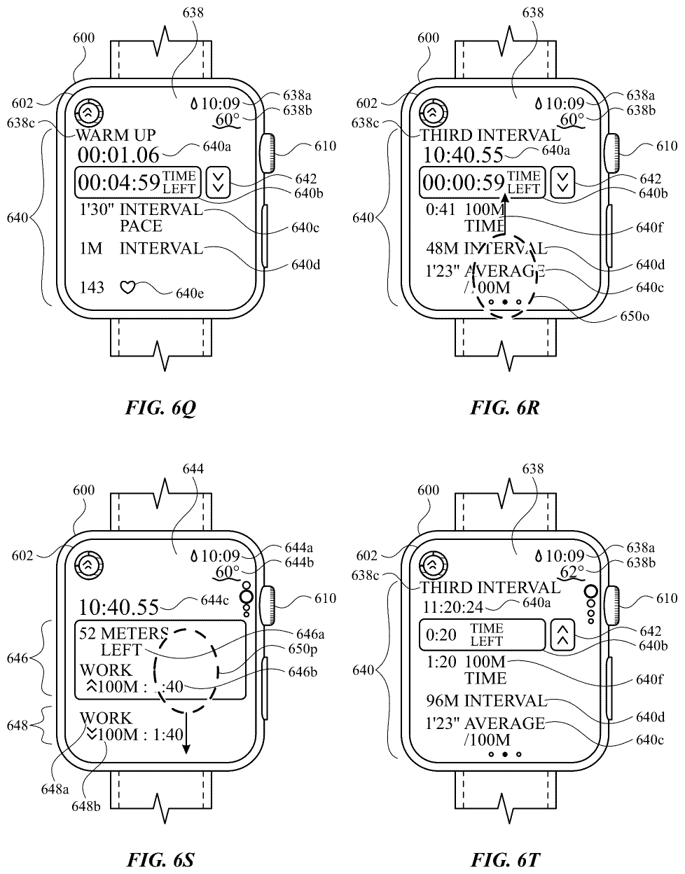

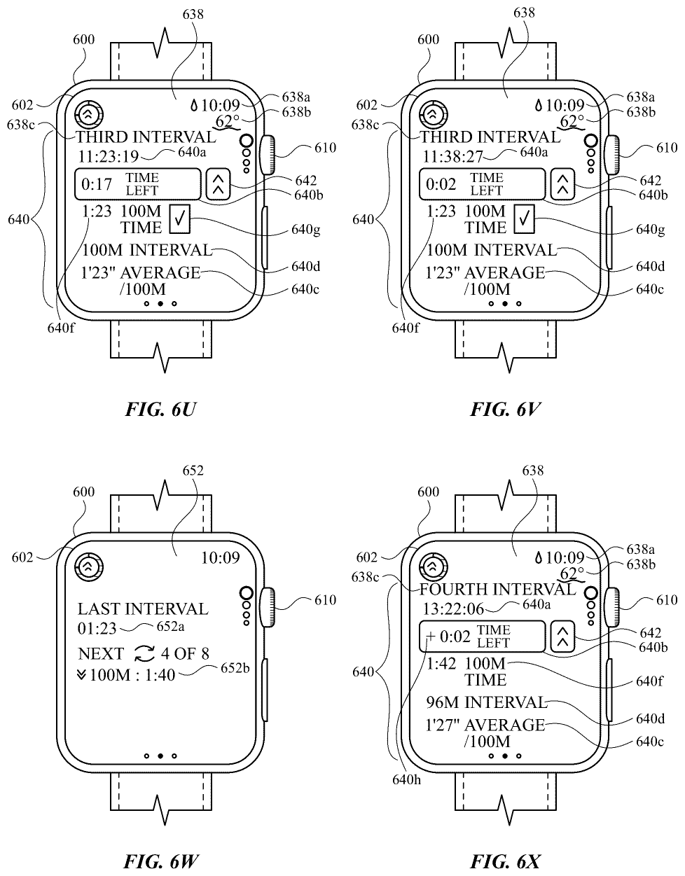

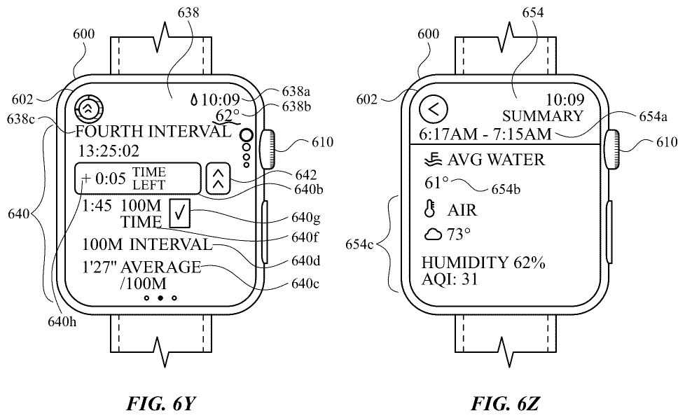

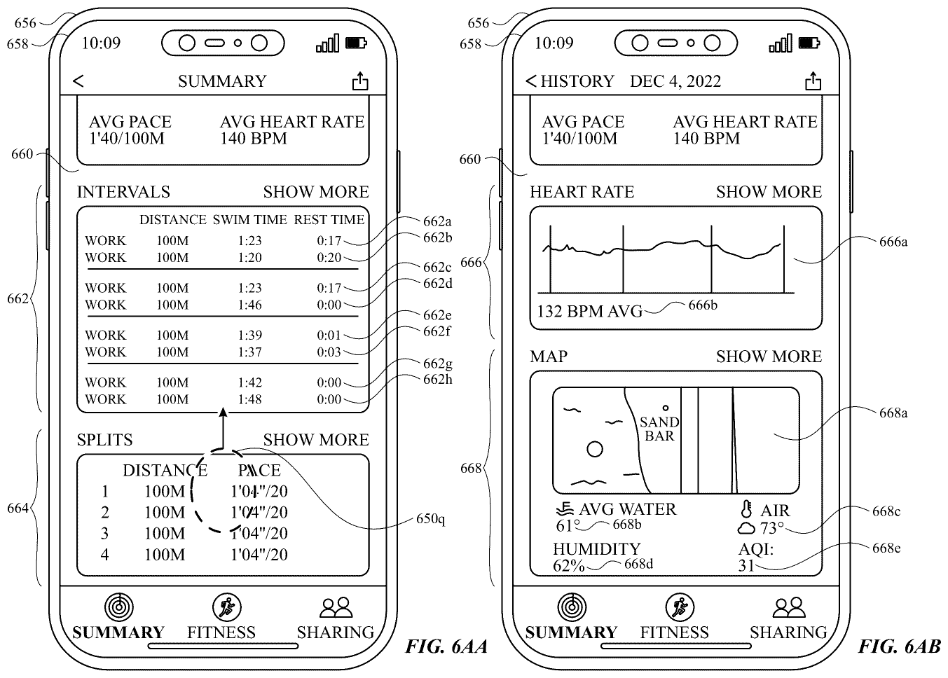

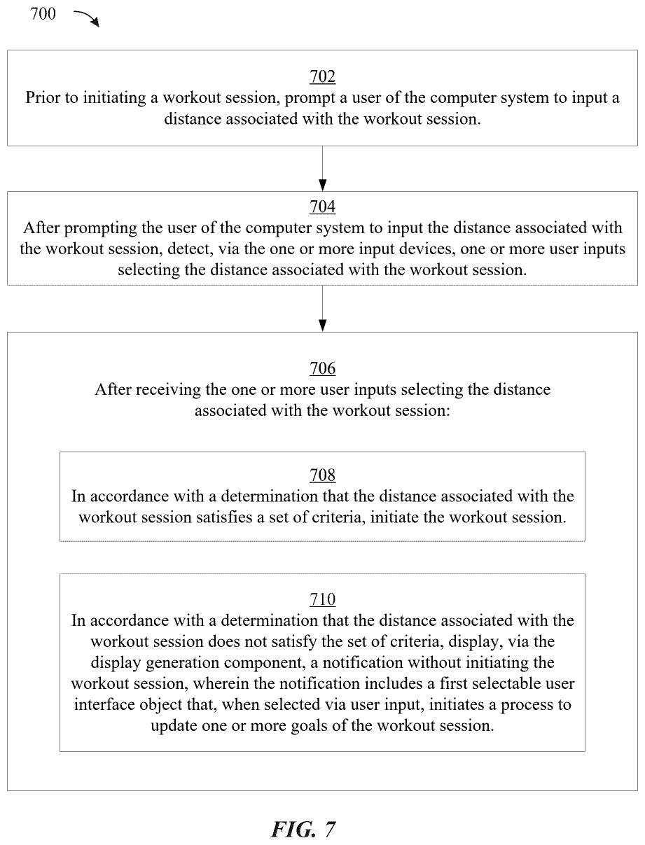

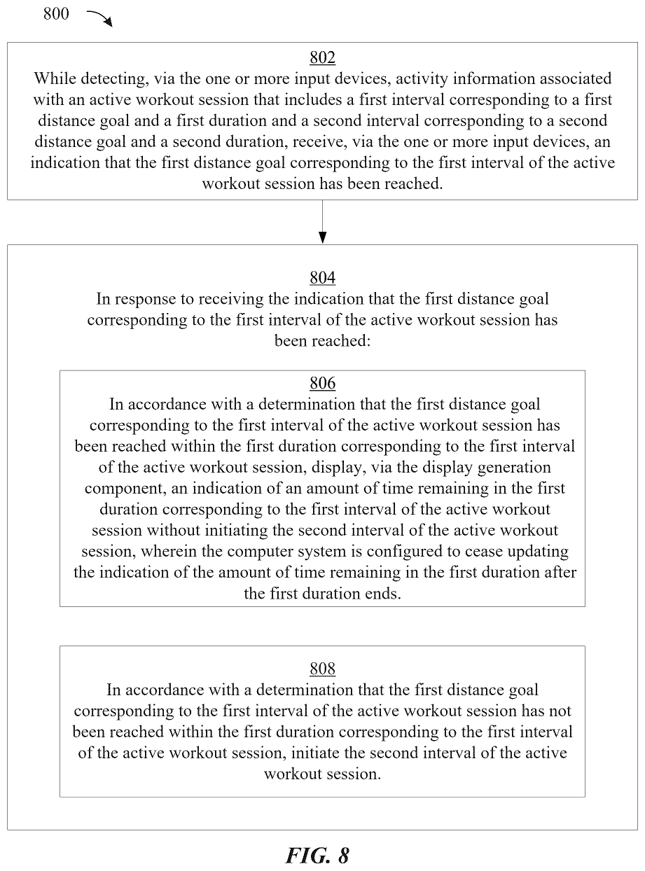

Below, FIGS. 1A-1B, 2, 3A-3G, 4A-4B, and 5A-5B provide a description of exemplary devices for performing the techniques for managing event notifications. FIGS. 6A-6AB illustrate exemplary user interfaces for managing workout sessions. FIG. 7 is a flow diagram illustrating methods of updating one or more goals of workout sessions in accordance with some embodiments. FIG. 8 is a flow diagram illustrating methods of displaying information about respective intervals of workout sessions. The user interfaces in FIGS. 6A-6AB are used to illustrate the processes described below, including the processes in FIGS. 7 and 8.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HMD), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

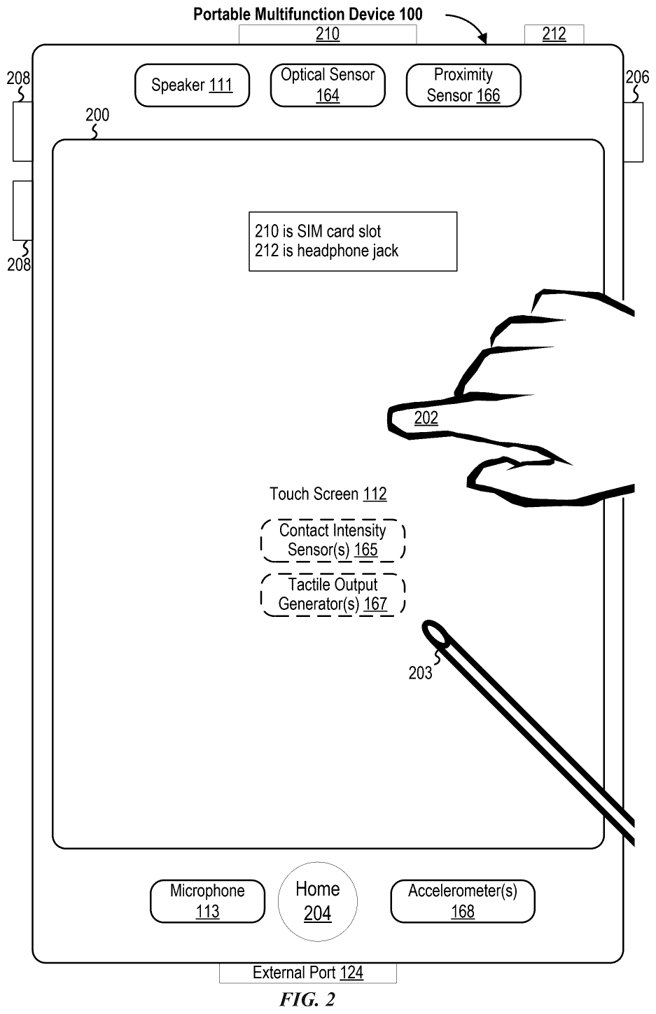

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

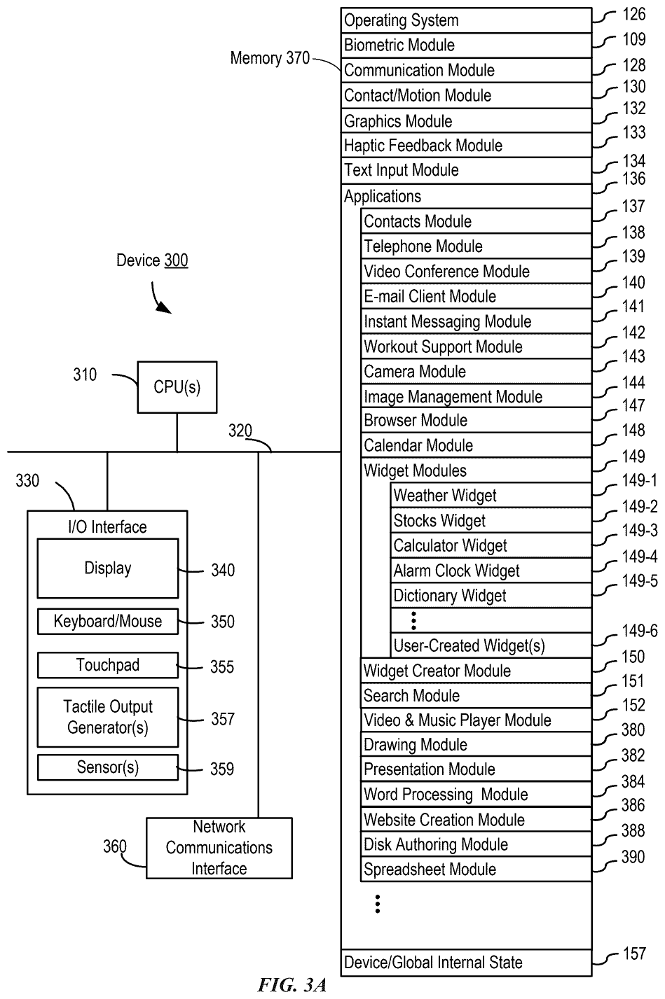

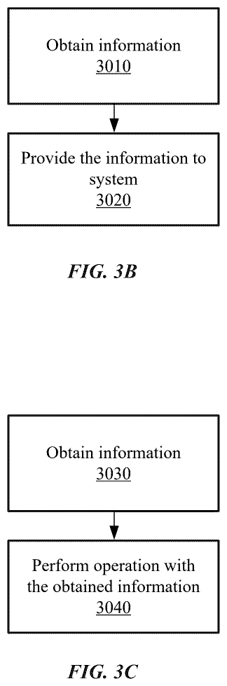

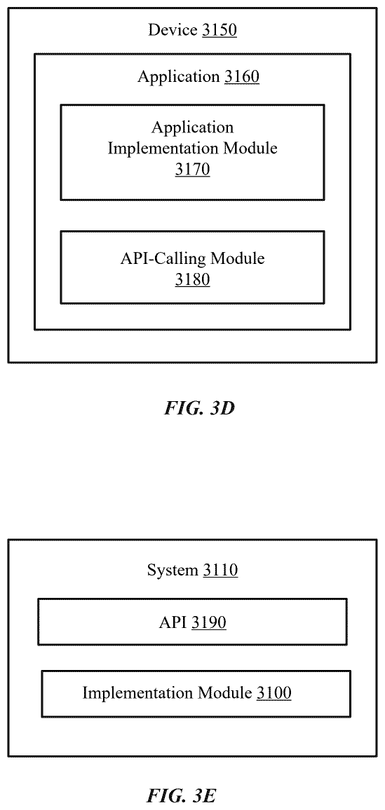

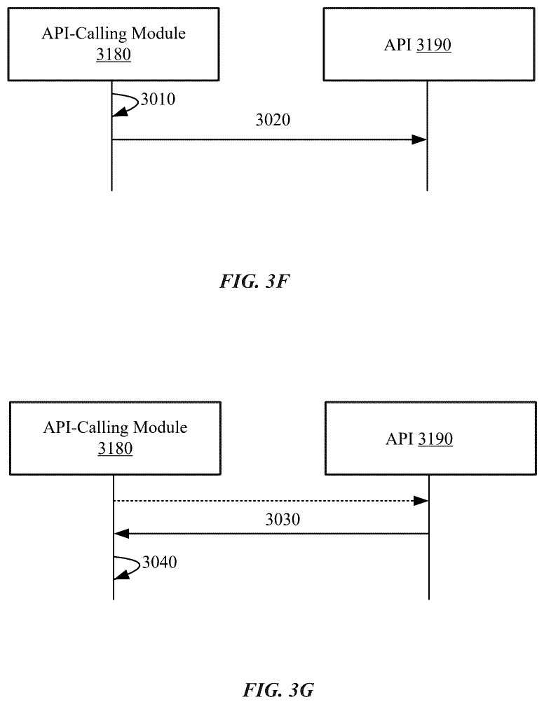

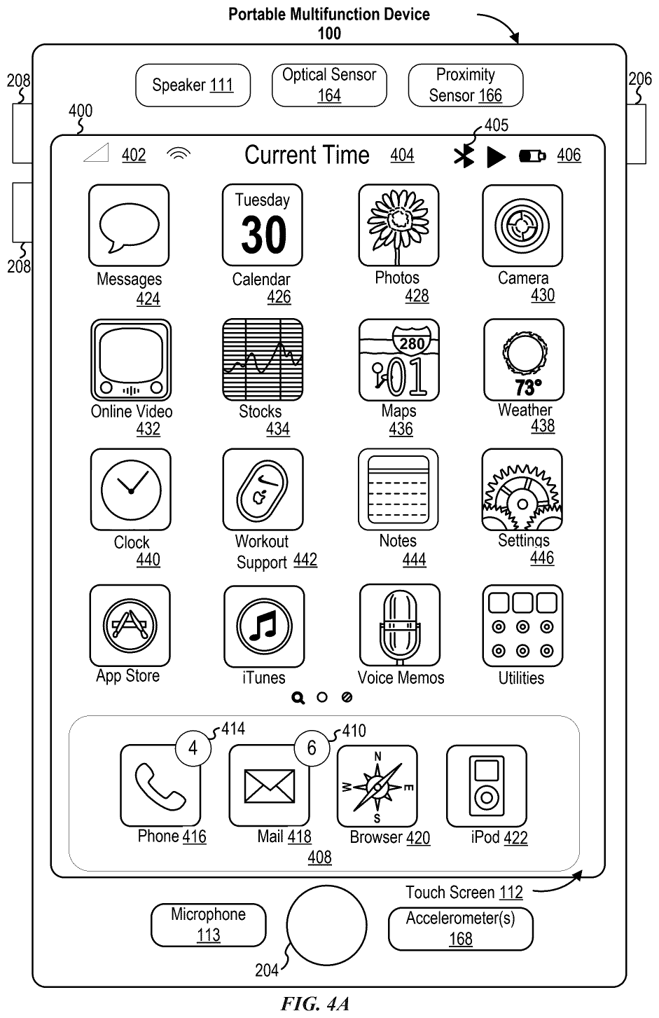

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HMD) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform methods 700 and/or 800 (FIGS. 7 and/or 8) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

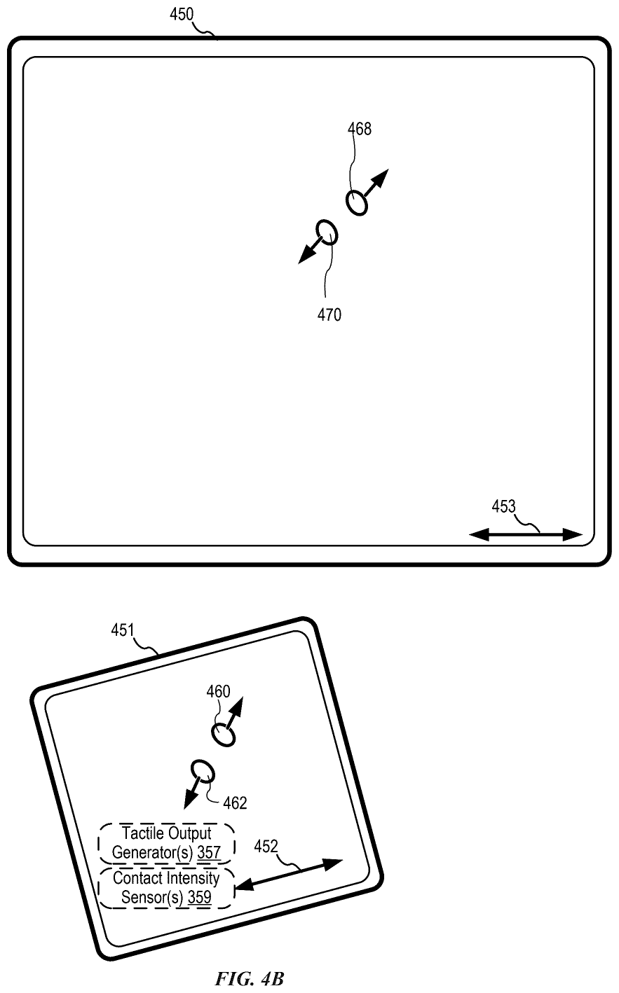

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

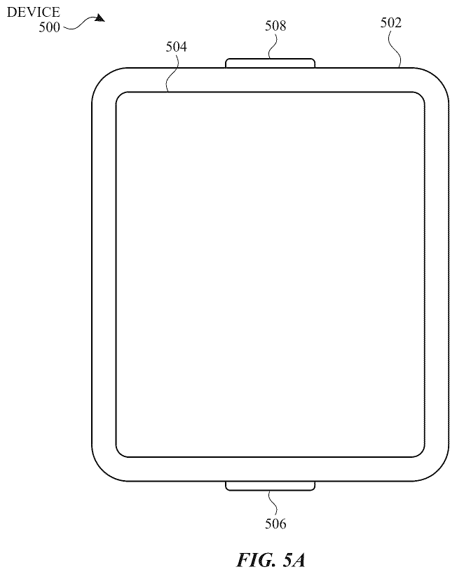

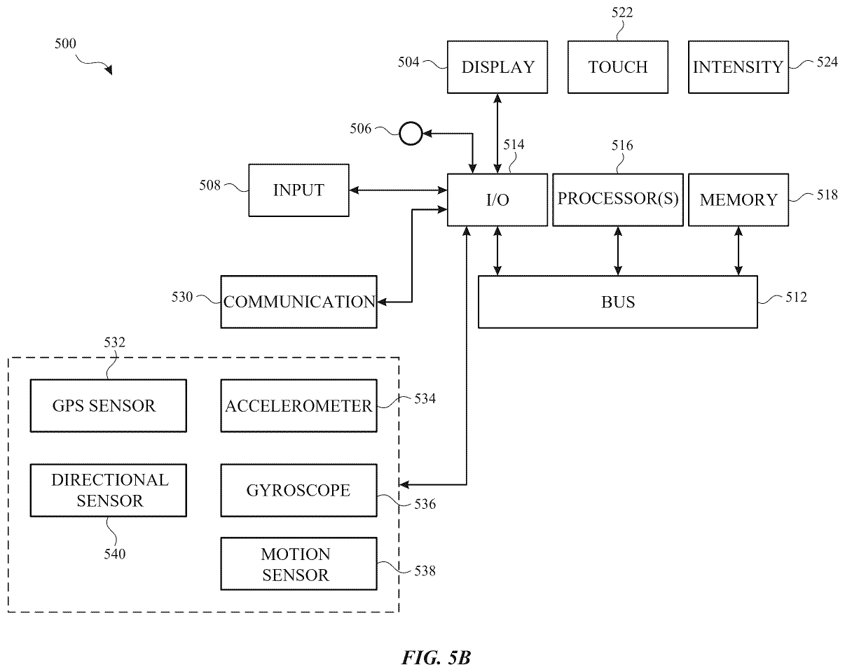

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700 and 800 (FIGS. 7 and 8). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6AB illustrate exemplary user interfaces for managing workout sessions, in accordance with some embodiments. The user interfaces in these figures are used to illustrate the processes described below, including the processes in FIGS. 7 and 8.

FIG. 6A illustrates computer system 600 displaying, via display 602, workout user interface 604. Workout user interface 604 includes pool swim workout option 606 and outdoor run workout option 608. In some embodiments, computer system 600 displays additional workout options on workout user interface 604 in response to detecting one or more user inputs (e.g., a swipe input and/or a rotational input on crown 610). In some embodiments, in response to detecting user input corresponding to a respective workout option 606 and/or 608, computer system 600 initiates a workout session for a workout corresponding to the respective workout option 606 and/or 608. In some embodiments, computer system 600 detects and/or receives information about physical activity performed by a user of computer system 600 and/or displays the information via display 602.

At FIG. 6A, pool swim workout option 606 includes settings user interface object 606a. As set forth below, in response to detecting user input corresponding to settings user interface object 606a, computer system 600 displays settings user interface 612 for the pool swim workout. Outdoor run workout option 608 includes settings user interface object 608a. In some embodiments, in response to detecting user input selecting settings user interface object 608a, computer system 600 displays a settings user interface for the outdoor run workout.

At FIG. 6A, computer system 600 detects user input 650a corresponding to selection of settings user interface object 606a of pool swim workout option 606. In response to detecting user input 650a, computer system 600 displays settings user interface 612, as shown at FIG. 6B.

At FIG. 6B, settings user interface 612 includes suggested workout region 614 and create workout user interface object 612a. Suggested workout region 614 includes user interface object 614a corresponding to a suggested pool swim workout. At FIG. 6A, user interface object 614a corresponds to a timed pool swim workout that lasts for 30 minutes. In some embodiments, in response to detecting user input corresponding to selection of user interface object 614a displayed in suggested workout region 614, computer system 600 initiates a workout session for the timed pool swim workout corresponding to user interface object 614a. In some embodiments, computer system 600 detects and/or receives information about physical activity performed by the user of computer system 600 over the course of 30 minutes while a user swims in a pool.

At FIG. 6B, computer system 600 detects user input 650b corresponding to selection of create workout user interface object 612a. In response to detecting user input 650b, computer system displays first workout creation user interface 616, as shown at FIG. 6C.

At FIG. 6C, first workout creation user interface 616 includes workout type user interface objects 616a-616d that correspond to respective types of swimming workouts. For instance, workout type user interface object 616a corresponds to an open swimming workout that does not include a distance goal and does not include a time goal. Workout type user interface object 616b corresponds to a distance swimming workout that includes a distance goal, but not a time goal. Workout type user interface object 616c corresponds to a timed swimming workout that includes a time goal and/or duration for the swimming workout, but not a distance goal. Workout type user interface object 616d corresponds to a time cycle swimming workout that includes different intervals and/or portions having respective distance goals and/or time goals.

At FIG. 6C, computer system 600 detects user input 650c corresponding to selection of workout type user interface object 616d. In response to detecting user input 650c, computer system 600 displays second workout creation user interface 618, as shown at FIG. 6D.

Second workout creation user interface 618 includes warmup user interface object 618a, interval user interface object 618b, and add user interface object 618c. Warmup user interface object 618a corresponds to a warmup portion of the time cycle swimming workout. In some embodiments, in response to detecting user input corresponding to warmup user interface object, computer system 600 displays one or more options for customizing the warmup portion of the workout. For instance, in some embodiments, computer system 600 displays one or more options to adjust a duration of the warmup portion, adjust a distance of the warmup portion, and/or remove the warmup portion from the time cycle swimming workout.

Interval user interface object 618b corresponds to an interval portion of the time cycle swimming workout. At FIG. 6D, interval user interface object 618b indicates that the interval portion of the time cycle swimming workout includes eight intervals, where each interval includes a distance goal of 100 meters and a time goal (e.g., duration) of one minute and 40 seconds. In some embodiments, computer system 600 displays interval user interface object 618b in response to detecting user input 650c, such that interval user interface object 618b is a default and/or suggested portion of the time cycle swimming workout. In some embodiments, computer system 600 forgoes display of interval user interface object 618b in response to detecting user input 650c and displays interval user interface object 618b after (e.g., in response to) detecting one or more user inputs. For instance, in some embodiments, computer system detects one or more user inputs requesting to add the portion of the time cycle swimming workout corresponding to interval user interface object 618b. In some embodiments, the one or more user inputs requesting to add the portion of the time cycle swimming workout corresponding to interval user interface object 618b include user inputs selecting, customizing, and/or otherwise choosing various characteristics of the portion of the time cycle swimming workout. In some embodiments, in response to detecting user input corresponding to interval user interface object 618b, computer system 600 displays one or more options for customizing and/or modifying the interval portion of the time cycle swimming workout. For instance, in some embodiments, computer system 600 displays one or more options to adjust a number of intervals in the interval portion, adjust a distance goal of one or more respective intervals, adjust a time goal (e.g., duration) of one or more respective intervals, and/or remove the interval portion of the time cycle swimming workout.

In some embodiments, computer system 600 displays additional user interface objects corresponding to different sections of a time cycle swimming workout on second workout creation user interface 618. In some embodiments, computer system 600 displays additional user interface objects corresponding to different portions of the time cycle swimming workout in response to user input, such as a swipe gesture and/or a rotation of crown 610.

At FIG. 6D, computer system 600 detects user input 650d corresponding to add user interface object 618c. In some embodiments, in response to detecting add user input 650d, computer system 600 displays a third workout creation user interface that includes one or more selectable options for selecting, modifying, and/or customizing a new portion of the time cycle swimming workout. For instance, in some embodiments, computer system 600 displays one or more selectable options corresponding to one or more portion types (e.g., intervals, stroke type, distance goal, and/or time goal), a number of intervals and/or repetitions, one or more swimming stroke types, and/or one or more goals for the new portion of the time cycle swimming workout. As such, a user of computer system 600 can add portions to the time cycle swimming workout in order to customize the time cycle swimming workout based on their fitness level.

At FIG. 6E, computer system 600 has detected one or more user inputs requesting to add a kick swim portion to the time cycle swimming workout, as indicated by kick swim user interface object 618d. At FIG. 6E, computer system 600 has added the kick swim portion to the time cycle swimming workout (e.g., in response to the one or more user inputs requesting to add a kick swim portion to the time cycle swimming workout). Kick swim user interface object 618d indicates that the kick swim portion includes a swim stroke type of kick swim and a distance goal of 25 meters. In some embodiments, kick swim user interface object 618d indicates a time goal in response to computer system 600 detecting one or more user inputs selecting a duration in which the user intends to complete the 25 meter kick swim.

While FIG. 6E illustrates computer system 600 adding kick swim user interface object 618d, computer system 600 is configured to add and display additional user interface objects corresponding to respective portions of the time cycle swimming workout based on one or more user inputs corresponding to add user interface object 618c.

At FIG. 6E, computer system 600 detects user input 650e directed to second workout creation user interface 618. In response to detecting user input 650e, computer system scrolls and/or translates second workout creation user interface 618, as shown at FIG. 6F.

At FIG. 6F, computer system 600 displays workout information 618e and create workout user interface object 618f of second workout creation user interface 618. In some embodiments, workout information 618e includes details and/or guidance about one or more features of the time cycle swimming workout and/or user interfaces that computer system 600 is configured to display when computer system 600 initiates a workout session for the time cycle swimming workout. At FIG. 6F, computer system 600 detects user input 650f corresponding to create workout user interface object 618f. In response to detecting user input 650f, computer system 600 displays settings user interface 612, as shown at FIG. 6G.

At FIG. 6G, computer system 600 displays time cycle user interface object 614b in suggested workout region 614 of settings user interface 612 in response to detecting user input 650f creating the time cycle swimming workout. In some embodiments, computer system 600 displays user interface objects corresponding to workouts created in suggested workout region 614 of settings user interface 612. In some embodiments, computer system 600 displays user interface objects corresponding to workouts created within a threshold duration from a current time (e.g., within one day, within one week, within two weeks, within one month, or within 6 months) in suggested workout region 614 of settings user interface 612. In some embodiments, computer system 600 displays user interface objects corresponding to workouts initiated by computer system 600 (and, optionally, performed by the user) within a threshold duration from a current time (e.g., within one day, within one week, within two weeks, within one month, or within 6 months) in suggested workout region 614 of settings user interface 612.

At FIG. 6G, computer system detects user input 650g corresponding to time cycle user interface object 614b. In response to detecting user input 650g, computer system 600 displays distance user interface 620, as shown at FIG. 6H.

Distance user interface 620 prompts a user of computer system 600 to provide a distance of a pool length in which the user intends to perform the time cycle swimming workout corresponding to time cycle user interface object 614b. For instance, distance user interface 620 includes indicator 620a that provides the user of computer system 600 with an indication that computer system 600 is prompting the user for a pool length. Distance user interface 620 includes distance selection user interface object 620b that includes a plurality of numerals corresponding to respective distances for a pool length. In some embodiments, computer system 600 scrolls and/or translates the plurality of numerals of distance selection user interface object 620b in response to user input, such as a swipe gesture, a rotation of crown 610, and/or an air gesture. At FIG. 6H, distance user interface 620 includes unit selection user interface object 620c indicating that a currently selected unit corresponding to the currently selected distance is meters. In some embodiments, computer system 600 displays one or more selectable options corresponding to respective units of distance in response to detecting user input corresponding to unit selection user interface object 620c (e.g., the computer system displays a drop-down menu and/or another user interface). In some embodiments, computer system 600 adjusts and/or changes a currently selected unit of distance to a next unit of distance in response to detecting user input corresponding to unit selection user interface object 620c.

At FIG. 6H, distance user interface 620 includes start user interface object 620d for initiating the time cycle swimming workout (e.g., after computer system 600 detects one or more user input selecting the pool length distance). For instance, at FIG. 6H, computer system 600 detects user input 650h corresponding to selection of start user interface object 620d. In response to detecting user input 650h, computer system 600 compares a selected pool length distance to one or more distance goals of the time cycle swimming workout. When computer system 600 determines that the selected pool length distance is consistent with the one or more distance goals of the time cycle swimming workout, computer system 600 initiates the time cycle swimming workout, as shown at FIG. 6I.

As set forth below with reference to FIGS. 6J-6O, when computer system 600 determines that the selected pool length distance is not consistent with at least one distance goal of the one or more distance goals of the time cycle swimming workout, computer system 600 displays a prompt (e.g., prompt 624 and/or prompt 630). In some embodiments, the prompt notifies the user of computer system 600 that the selected pool length distance is not consistent with at least one distance goal of the one or more distance goals, enables a user to update the at least one distance goal of the one or more distance goals, and/or enables computer system 600 to provide suggestions for updating the at least one distance goal of the one or more distance goals. In some embodiments, a distance goal of the time cycle swimming workout is not consistent with the selected pool length distance when the distance goal includes a unit of distance that is different from a unit of distance corresponding to the selected pool length distance. In some embodiments, a distance goal of the time cycle swimming workout is not consistent with the selected pool length distance when the selected pool length distance is not divisible by the distance goal (e.g., even when the selected pool length distance and the distance goal include the same unit of distance).

In some embodiments, computer system 600 determines that the selected pool length distance is not consistent with at least one goal of a swimming workout by identifying one or more (e.g., each) goal of the swimming workout and determining whether the one or more (e.g., each) goal includes a swim distance and/or a swim distance and time. In some embodiments, when computer system 600 determines that one or more goals of the swimming workout include a swim distance and/or a swim distance and time, computer system 600 compares the swim distance to the selected pool length distance. In some embodiments, when one or more goals of the swimming workout include a swim distance (and/or a swim distance portion of the swim distance and time) that is in different units than the units of the selected pool length distance, computer system 600 converts the swim distance to the units of the selected pool length distance to suggest and/or recommend a new swim distance. In some embodiments, converting the swim distance to the units of the selected pool length distance includes replacing the units of the swim distance with the units of the selected pool length distance (e.g., changing 25 yards to 25 meters or changing 50 meters to 50 yards). In some embodiments, converting the swim distance to the units of the selected pool length distance includes a calculation of the swim distance in its current units to the units of the selected pool length distance (e.g., converting 25 yards to 22.86 meters or converting 50 meters to 54.68 yards). In some embodiments, computer system 600 converts the swim distance and/or the pool length distance to have the same unit of measure (e.g., meters, yards, or feet).

In some embodiments, computer system 600 compares the one or more goals that include a swim distance to the selected pool length distance to determine (and/or suggest) new swim distances (e.g., to replace and/or use instead of the original swim distance of the goal) based on the selected pool length distance. For instance, a lap of a pool includes a user swimming one length of the pool, and thus, includes the user swimming the selected pool length distance one time. In some embodiments, the swim distance of one or more goals of the swimming workout is not divisible by the selected pool length distance, such that the number of laps the user would need to swim to reach the swim distance would not be a whole number. In some embodiments, computer system 600 determines the new swim distance so that it is divisible by the selected pool length distance. For example, in some embodiments, computer system determines the new swim distance using Equations 1 and 2, set forth below. In some embodiments, computer system 600 uses Equation 1 to determine a number of laps associated with the swim distance by rounding (e.g., rounding to the nearest whole number) the quotient of the swim distance divided by the selected pool length distance. In some embodiments, computer system 600 uses Equation 2 to determine the new swim distance by multiplying the number of laps (e.g., the number of laps determined using Equation 1) by the selected pool length distance.

$$Number of Laps = Round ( \frac{Original Distance}{Pool Length} ) \qquad Eq . 1$$

$$New Distance = Number of Laps * Pool Length \qquad Eq . 2$$

In some embodiments, the swimming workout includes one or more goals that include a swim distance and time. For instance, the swimming workout may include a goal to swim 40 meters in 1 minute and 40 seconds. However, in some embodiments, the swim distance portion of the distance and time goal includes units that are different from the unit of the selected pool length distance and/or includes a swim distance that is not divisible by the selected pool length distance. In some embodiments, computer system 600 determines both a new swim distance and a new time for the distance and time goal of the swimming workout. In some embodiments, computer system 600 determines the new swim distance for the distance and time goal using Equations 1 and 2, as shown above. In some embodiments, computer system 600 determines the new time using Equations 3 and 4, set forth below. For instance, in some embodiments, computer system 600 determines an original speed associated with the distance and time goal by dividing the swim distance by the time of the distance and time goal, as shown at Equation 3. In some embodiments, computer system 600 determines the new time by dividing the new swim distance (e.g., the new swim distance determined by Equations 1 and 2) by the original speed (e.g., the original speed determined by Equation 3), as shown at Equation 4. Thus, computer system 600 provides new suggested goals that are consistent with the selected pool length distance and the original goals of the swimming workout.

$$Original Speed = \frac{Original Distance}{Original Time} \qquad Eq . 3$$

$$New Time = \frac{New Distance}{Original Speed} \qquad Eq . 4$$

In some embodiments, the swim distance of a goal of the swimming workout is less than the selected pool length distance. In some embodiments, when the swim distance of the goal of the swimming workout is less than the selected pool length distance, computer system 600 determines the new swim distance for the goal of the swimming workout as the selected pool length distance (e.g., so that the user is not swimming only a portion of the pool and/or a portion of a lap). In some embodiments, the computer system suggests the new swim distance for the goal of the swimming workout as the selected pool length distance even when the selected pool length distance is relatively long when compared to the swim distance of the goal (e.g., the pool length is 50 meters and the swim distance is 25 yards).

In some embodiments, the swimming workout includes one or more repeat goals that include swim distances. For instance, in some embodiments, the swimming workout includes a goal to repeat a particular swimming stroke for the swim distance a certain number of times. In some embodiments, computer system 600 determines a new number of repetitions for the repeat workout based on the selected pool length distance. For instance, in some embodiments, the swimming workout includes a repeat goal to swim 25 yards four times and the selected pool length distance is 50 meters. In some embodiments, computer system 600 determines the new swim distance as 50 meters and determines the new number of repetitions as 2 instead of 4. Thus, computer system 600 proposes and/or suggests new goals for the swimming workout based on the selected pool length distance so that the new proposed and/or suggested goals are closely aligned with the original goals of the swimming workout.

At FIG. 6I, computer system 600 initiates the time cycle swimming workout and displays countdown user interface 622. Countdown user interface 622 provides a countdown for a duration of time before computer system 600 begins tracking, detecting, and/or determining information about the time cycle swimming workout. For instance, countdown user interface 622 includes progress indicator 622a that indicates how much time remains in the countdown and/or an amount of time until computer system 600 begins tracking, detecting, and/or determining information about the time cycle swimming workout. At FIG. 6I, countdown user interface 622 includes numeral indicator 622b that indicates a number of seconds remaining in the countdown and/or an amount of seconds remaining until computer system 600 begins tracking, detecting, and/or determining information about the time cycle swimming workout. In some embodiments, computer system 600 animates countdown user interface 622, such that an appearance of progress indicator 622a and/or numeral indicator 622b change over time. As set forth below with respect to FIG. 6Q, computer system 600 displays workout user interface 638 after the duration of time of the countdown of countdown user interface 622 elapses and/or expires.

At FIG. 6J, computer system 600 displays distance user interface 620 before initiating the workout session of the time cycle swimming workout (e.g., in response to and/or after detecting user input 650g). At FIG. 6J, distance selection user interface object 620b and unit selection user interface object 620c indicate that a pool length of 20 meters is currently selected. For instance, in some embodiments, computer system 600 receives one or more user inputs directed to distance selection user interface object 620b requesting to select the number 20.

However, as set forth above, the time cycle swimming workout includes a kick swim portion that has a distance goal of 25 meters. Because a distance of 25 meters is not divisible by a distance of 20 meters, the distance goal of the kick swim portion is inconsistent with the currently selected pool length distance. At FIG. 6J, computer system 600 detects user input 650i corresponding to selection of start user interface object 620d. In response to detecting user input 650i and based on the determination that the distance goal of the kick swim portion is inconsistent with the currently selected pool length distance, computer system 600 displays prompt 624, as shown at FIG. 6K.

At FIG. 6K, prompt 624 includes information 624a that notifies a user of computer system 600 that the distance goal of the kick swim portion is inconsistent with the selected pool length distance. For instance, information 624a includes text describing that the time cycle swimming workout includes a portion (e.g., the kick swim portion) having a distance goal of 25 meters, which does not match the selected pool length distance of 20 meters. In other words, in order for the user of computer system 600 to reach the distance goal of 25 meters, the user would have to traverse a full length of the pool followed by 5 meters (e.g., portion of a length of the pool). Prompt 624 includes change user interface object 624b that enables the user to update the distance goal of the kick swim portion to be consistent with the selected pool length distance. Prompt 624 also includes dismiss user interface object 624c. At FIG. 6K, computer system 600 detects user input 650j corresponding to selection of dismiss user interface object 624c. In some embodiments, in response to detecting user input 650j corresponding to dismiss user interface object 624c, computer system 600 displays settings user interface 612 and/or distance user interface 620. In some embodiments, in response to detecting user input 650j corresponding to dismiss user interface object 624c, computer system 600 initiates a workout session for the time cycle swimming workout without updating the distance goal for the kick swim portion and/or any other portions of the time cycle swimming workout that are inconsistent with the selected pool length distance.

Additionally or alternatively, at FIG. 6K, computer system 600 detects user input 650k corresponding to selection of change user interface object 624b. In response to detecting user input 650k, computer system 600 displays goal user interface 626, as shown at FIG. 6L. At FIG. 6L, goal user interface 626 includes warmup user interface object 626a, interval user interface object 626b, kick swim user interface object 626c, and start user interface object 626d. At FIG. 6L, computer system 600 displays kick swim user interface object 626c with initial goal indicator 628a and suggested goal indicator 628b because the distance goal for the kick swim portion is not consistent with the selected pool length distance (e.g., 25 meters is not divisible by 20 meters or vice versa). Computer system 600 displays initial goal indicator 628a with a strikethrough text effect and displays suggested goal indicator 628b without the strikethrough text effect to suggest a change to the distance goal of the kick swim portion. For instance, the suggested distance goal corresponding to suggested goal indicator 628b is 20 meters, which is divisible by the selected pool length distance of 20 meters. As such, computer system 600 provides a suggested change to the distance goal based on the selected pool length distance.

In some embodiments, computer system 600 displays another user interface and/or one or more user interface objects in response to detecting selection of kick swim user interface object 626c. For instance, in some embodiments, in response to detecting user input corresponding to kick swim user interface object 626c, computer system 600 displays a user interface and/or other suggested distance goals for the kick swim portion of the time cycle swimming workout so that the user of computer system 600 can select an updated distance goal that is consistent with the selected pool length distance and the fitness level of the user.

At FIG. 6L, computer system 600 does not display warmup user interface object 626a with a suggested goal indicator because the warmup portion of the time cycle swimming workout does not include a distance goal and/or a distance goal that is inconsistent with the selected pool length distance. Similarly, computer system 600 does not display interval user interface object 626b with a suggested goal indicator because a distance goal of the interval portion of the time cycle swimming workout includes a distance that is divisible by the selected pool length distance (e.g., 100 meters can be reached by swimming the length of a 20 meter pool five times). Thus, computer system 600 suggests updated distance goals for portions of a workout that include distances that are inconsistent with the selected pool length distance.

In some embodiments, in response to detecting user input corresponding to start user interface object 626d, computer system 600 initiates the time cycle swimming workout with an updated distance goal for the kick swim portion of 20 meters instead of 25 meters. In some embodiments, in response to detecting user input corresponding to start user interface object 626d, computer system 600 displays countdown user interface 622, as shown at FIGS. 6I and/or 6P.

At FIG. 6M, computer system 600 displays distance user interface 620 before initiating the workout session of the time cycle swimming workout (e.g., in response to and/or after detecting user input 650g). At FIG. 6M, distance selection user interface object 620b and unit selection user interface object 620c indicate that a pool length of 25 yards is currently selected. For instance, in some embodiments, computer system 600 receives one or more user inputs directed to distance selection user interface object 620b requesting to select the number 25 and/or one or more user inputs directed to unit selection user interface object 620c requesting to change the distance unit to yards (e.g., change the currently selected distance unit from meters to yards).

However, as set forth above, the time cycle swimming workout includes an interval portion that has a distance goal of 100 meters and a kick swim portion that has a distance goal of 25 meters. Because meters and yards are not the same unit of distance, the distance goal of both the interval portion and the distance goal of the kick swim portion are inconsistent with the currently selected pool length distance. At FIG. 6M, computer system 600 detects user input 650l corresponding to selection of start user interface object 620d. In response to detecting user input 650l and based on the determination that the distance goal of the interval portion and the distance goal of the kick swim portion are inconsistent with the currently selected pool length distance, computer system 600 displays prompt 630, as shown at FIG. 6N.

At FIG. 6N, prompt 630 includes information 630a that notifies a user of computer system 600 that the distance goal of one or more portions of the time cycle swimming workout are inconsistent with the selected pool length distance. For instance, information 630a includes text indicating that a distance goal corresponding to at least one portion of the time cycle swimming workout includes a distance unit of meters, which does not match the selected distance unit for the selected pool length distance of yards. Prompt 630 includes change user interface object 630b that enables the user to update the one or more goals of the interval portion and/or the kick swim portion to be consistent with distance for the selected pool length distance. Prompt 630 also includes dismiss user interface object 630c. As set forth above, with reference to FIG. 6K, in some embodiments, in response to detecting user input corresponding to dismiss user interface object 630c, computer system 600 displays settings user interface 612 and/or distance user interface 620. In some embodiments, in response to detecting user input corresponding to dismiss user interface object 630c, computer system 600 initiates a workout session for the time cycle swimming workout without updating one or more goals for the interval portion, the kick swim portion, and/or any other portions of the time cycle swimming workout that are inconsistent with the distance unit for the selected pool length distance.

Additionally or alternatively, at FIG. 6N, computer system 600 detects user input 650m corresponding to selection of change user interface object 630b. In response to detecting user input 650m, computer system 600 displays goal user interface 632, as shown at FIG. 6O. At FIG. 6O, goal user interface 632 includes warmup user interface object 632a, interval user interface object 632b, kick swim user interface object 632c, and start user interface object 632d.

Interval user interface object 632b includes initial goal indicator 634a and suggested goal indicator 634b because the distance unit associated with the interval portion is inconsistent with the distance unit for the selected pool length distance (e.g., the initial goal includes a distance unit of meters and the selected pool length distance includes a distance unit of yards). At FIG. 6O, initial goal indicator 634a includes both an initial distance goal (“100M”) and an initial time goal and/or duration (“1:40”). Because one yard is less than one meter, computer system 600 provides both a suggested distance goal and a suggested time goal and/or duration via suggested goal indicator 634b. In other words, computer system 600 displays a suggested distance goal and a suggested time goal that are based on the initial distance goal, the initial time goal, and/or the selected pool length distance. In some embodiments, computer system 600 determines the suggested distance goal based on the selected pool length distance and/or the initial distance goal. At FIG. 6O, computer system 600 provides a suggested distance goal of 100 yards instead of 100 meters. In some embodiments, computer system 600 determines the suggested time goal based on the suggested distance goal, the initial distance goal, the initial time goal, and/or the selected pool length distance. As such, computer system 600 provides suggested distance and time goals for the interval portion of the time cycle swimming workout that includes a pace (e.g., distance over time) that is consistent with the initial distance goal and the initial time goal.

At FIG. 6O, computer system 600 displays initial goal indicator 634a with a strikethrough text effect and displays suggested goal indicator 634b without the strikethrough text effect to suggest a change to the distance goal and the time goal for the interval portion of the time cycle swimming workout. For instance, the suggested distance goal corresponding to suggested goal indicator 634b is 100 yards, which is divisible by the selected pool length distance of 25 yards and similar in distance to the initial distance goal of 100 meters. In addition, the suggested time goal is one minute and 31 seconds, which is based on an initial pace goal that is determined from the initial distance goal and the initial time goal. As such, computer system 600 provides a suggested change to the goals of the interval portion based on the distance unit for selected pool length distance.

In some embodiments, computer system 600 displays another user interface and/or one or more user interface objects in response to detecting selection of interval user interface object 632b. For instance, in some embodiments, in response to detecting user input corresponding to interval user interface object 632b, computer system 600 displays a user interface and/or other suggested goals for the interval portion of the time cycle swimming workout so that the user of computer system 600 can select one or more updated goals that are consistent with the distance unit for the selected pool length distance and the fitness level of the user.

At FIG. 6O, computer system 600 displays kick swim user interface object 632c with initial goal indicator 636a and suggested goal indicator 636b because distance unit of the distance goal for the kick swim portion is not consistent with distance unit of the selected pool length distance (e.g., the initial goal includes a distance unit of meters and the selected pool length distance includes a distance unit of yards). Computer system 600 displays initial goal indicator 636a with a strikethrough text effect and displays suggested goal indicator 636b without the strikethrough text effect to suggest a change to the distance goal of the kick swim portion based on the selected pool length distance. For instance, the suggested distance goal corresponding to suggested goal indicator 636b is 25 yards instead of 25 meters. As such, computer system 600 provides a suggested change to the distance goal based on the distance unit for selected pool length distance.

In some embodiments, computer system 600 displays another user interface and/or one or more user interface objects in response to detecting selection of kick swim user interface object 632c. For instance, in some embodiments, in response to detecting user input corresponding to kick swim user interface object 632c, computer system 600 displays a user interface and/or other suggested distance goals for the kick swim portion of the time cycle swimming workout so that the user of computer system 600 can select an updated distance goal that is consistent with the selected pool length distance and the fitness level of the user.

At FIG. 6O, computer system 600 does not display warmup user interface object 632c with a suggested goal indicator because the warmup portion of the time cycle swimming workout does not include a distance goal and/or a distance goal that is inconsistent with the selected pool length distance. Thus, computer system 600 suggests updated goals for portions of a workout that include inconsistencies with the selected pool length distance (e.g., distance goals having inconsistent distance units with the selected pool length distance and/or distance goals having distances that are not divisible by the selected pool length distance).

At FIG. 6O, computer system 600 detects user input 650n corresponding to selection of start user interface object 632d. In response to detecting user input 650n corresponding to start user interface object 632d, computer system 600 initiates the time cycle swimming workout with the suggested updated goals for the interval portion and the kick swim portion and displays countdown user interface 622, as shown at FIG. 6P.

At FIG. 6P, computer system 600 initiates the time cycle swimming workout and displays countdown user interface 622. As set forth above, countdown user interface 622 provides a countdown for a duration of time indicating when computer system 600 will begin tracking, detecting, and/or determining information about the time cycle swimming workout. For instance, countdown user interface 622 includes a progress indicator 622a that indicates how much time remains in the countdown and/or an amount of time until computer system 600 will begin tracking, detecting, and/or determining information about the time cycle swimming workout. Countdown user interface 622 includes numeral indicator 622b that indicates a number of seconds remaining in the countdown and/or an amount of second remaining until computer system 600 will begin tracking, detecting, and/or determining information of the time cycle swimming workout. In some embodiments, countdown user interface 622 is animated, such that computer system 600 changes and/or modifies an appearance of progress indicator 622a and/or numeral indicator 622b over time.

At FIG. 6Q, computer system 600 initiates the time cycle swimming workout and displays workout user interface 638 after the duration of time of the countdown of countdown user interface 622 elapses and/or expires. Workout user interface 638 includes time indicator 638a, water temperature indicator 638b, workout portion indicator 638c and workout information region 640. Time indicator 638a indicates a current time of day, such as a digital indication of the current time of day. Water temperature indicator 638b indicates a currently detected and/or determined water temperature in which computer system 600 is at least partially submerged. In some embodiments, computer system 600 includes a temperature sensor that is configured to provide information related to a temperature of an environment in which computer system 600 is positioned. In some embodiments, computer system 600 displays water temperature indicator 638b to indicate a currently detected and/or determined water temperature when computer system 600 is actively tracking and/or performing a swimming workout because computer system 600 is likely to be at least partially positioned within water during the swimming workout. In some embodiments, water temperature indicator 638b includes a real-time indication of the water temperature. In some embodiments, water temperature indicator 638b is configured to provide a detected and/or determined value of the water temperature at predefined periods of time (e.g., every second, every 5 seconds, every 10 seconds, every 30 seconds, every minute, or every 2 minutes). As set forth below with reference to FIGS. 6Z-6AB, in some embodiments, computer system 600 averages a plurality of detected and/or determined water temperature values over a duration of the time cycle swimming workout and displays an indication of the average water temperature on a workout summary user interface.

Workout portion indicator 638c of workout user interface 638 includes an indication, such as text, about the current portion of the time cycle swimming workout. For instance, at FIG. 6Q, workout portion indicator 638c includes the text “WARM UP” to indicate to a user of computer system 600 that the current portion of the time cycle swimming workout is the warmup portion of the time cycle swimming workout. In some embodiments, workout portion indicator 638c indicates a current portion of the time cycle swimming workout for which computer system 600 is tracking and/or detecting physical activity information. In some embodiments, workout portion indicator 638c notifies a user of computer system 600 that the workout information included in workout information region 640 of workout user interface 638 pertains to a particular portion of the time cycle swimming workout (e.g., the warmup portion).

Workout information region 640 includes overall duration indicator 640a, interval duration indicator 640b, interval pace indicator 640c, interval distance indicator 640d, and heart rate indicator 640e. Overall duration indicator 640a indicates a total amount of time since computer system 600 initiated the time cycle swimming workout and/or a total amount of time for which computer system 600 has been tracking and/or detecting physical activity information corresponding to the time cycle swimming workout. At FIG. 6Q, overall duration indicator 640a indicates that computer system 600 has recently initiated the time cycle swimming workout and that slightly more one second of time has elapsed. Thus, at FIG. 6Q, overall duration indicator 640a counts upward from 0 seconds to indicate the total amount of time and/or duration of the time cycle swimming workout. In some embodiments, such as when the active workout session includes a time goal and no distance goal, overall duration indicator 640a counts down from a time of the time goal to 0 seconds.

At FIG. 6Q, interval duration indicator 640b indicates an amount of time remaining in the current portion of the time cycle swimming workout (e.g., four minutes and 59 seconds). As set forth above, the warmup portion of the time cycle swimming workout includes a time goal of five minutes. As such, interval duration indicator 640b counts down from 5 minutes to 0 seconds to indicate an amount of time remaining in the warmup portion of the time cycle swimming workout. In some embodiments, such as when the warmup portion of the time cycle swimming workout includes a distance goal instead of a time goal, computer system 600 displays an indication of a remaining amount of distance of the distance goal instead of interval duration indicator 640b. At FIG. 6Q, computer system 600 displays interval user interface object 642 on workout user interface 638. In some embodiments, in response to detecting user input corresponding to selection of interval user interface object 642, computer system 600 displays information about a next portion and/or interval of the time cycle swimming workout and/or one or more additional portions and/or intervals of the time cycle swimming workout.

Interval pace indicator 640c indicates a currently detected and/or determined pace at which computer system 600 is moving over time. For instance, at FIG. 6Q, interval pace indicator 640c indicates that a currently detected and/or determined pace is one minute and 30 seconds to travel 100 meters. In some embodiments, interval pace indicator 640c includes a real time indication of a pace at which computer system 600 is moving over time. In some embodiments, computer system 600 updates interval pace indicator 640c at predefined times over the duration of the time cycle swimming workout (e.g., every second, every two seconds, every five seconds, every ten seconds, every 30 seconds, or every minute and/or when a distance goal is reached).

Interval distance indicator 640d indicates a distance at which computer system 600 has traveled, traversed, and/or moved over the course of the current interval, which is the warmup interval. As shown at FIG. 6Q, interval distance indicator 640d indicates that computer system 600 has moved a total distance of one meter in one second. Thus, computer system 600 includes one or more sensors that provide information about a distance in which computer system 600 has traveled, traversed, and/or moved over the course of the current interval. In some embodiments, computer system 600 displays, in addition to interval distance indicator 640d or in lieu of interval distance indicator 640d, an indication of a distance in which computer system 600 has traveled, traversed, and/or moved over the course of the entire active workout session.

At FIG. 6Q, heart rate indicator 640e provides an indication of a determined and/or detected heart rate of the user of computer system 600. In some embodiments, computer system 600 includes one or more sensors that provide information about a heart rate of the user of computer system 600. In some embodiments, heart rate indicator 640e is a real-time indication of a current heart rate of the user of computer system 600. In some embodiments, computer system 600 receives information about the heart rate of the user of computer system 600 at predefined periods of time (e.g., every second, every two seconds, every five seconds, every 10 seconds, every 30 seconds, or every minute).

Turning now to FIG. 6R, computer system 600 displays workout user interface 638 after the warmup portion of the time cycle workout has ended. For instance, workout user interface 638 indicates that the time cycle swimming workout is now in a third interval of an interval portion of the time cycle swimming workout. Workout portion indicator 638c includes text indicating that computer system 600 is tracking and/or detecting physical activity information related to a third interval of the interval portion of the time cycle swimming workout. In addition, workout information region 640 includes overall duration indicator 640a indicating that ten minutes and 40 seconds have elapsed since computer system 600 initiated the time cycle swimming workout.

Workout information region 640 further includes interval duration indicator 640b indicating that 59 seconds remain in the third interval of the interval portion of the time cycle swimming workout. As set forth above, the interval portion of the time cycle swimming workout includes eight intervals, where each interval includes a distance goal of 100 meters and a time goal of one minute and 40 seconds. Interval duration indicator 640b indicates that 59 seconds remain in the one minute and 40 seconds time goal and/or that 41 seconds have elapsed within the third interval of the interval portion of the time cycle swimming workout.

At FIG. 6R, workout information region 640 includes distance goal duration indicator 640f indicating an amount of time between a start of a current interval (e.g., the third interval) and a current time. In other words, distance goal duration indication 640f provides an indication of an amount of time computer system 600 has spent tracking whether computer system 600 has traveled, traversed, and/or moved for a distance corresponding to the distance goal of the current interval. At FIG. 6R, distance goal duration indicator 640f indicates that computer system 600 has been tracking and/or detecting a distance of movement of computer system 600 for 41 seconds. At FIG. 6R, distance goal duration indicator 640f counts upward from 0 seconds while computer system tracks and/or detects the distance of movement of computer system 600 for the current interval. As set forth below, in some embodiments, computer system 600 stops counting up distance goal duration indicator 640f when computer system 600 detects that the distance goal for the current interval has been reached (e.g., computer system 600 detects and/or determines that computer system has traveled, traversed, and/or moved a distance corresponding to the distance goal for the current interval).

Workout information region 640 includes interval distance indicator 640d indicating that computer system 600 has traveled, traversed, and/or moved a distance of 48 meters during the current interval (e.g., the third interval). In other words, interval distance indicator 640d indicates that computer system 600 has traveled, traversed, and/or moved a distance of 48 meters over 41 seconds (e.g., the amount of time elapsed during the current interval).

At FIG. 6R, workout information region 640 includes interval pace indicator 640c indicating a pace at which computer system 600 has moved over a duration of the current interval. Interval pace indicator 640c indicates a detected and/or determined rate of movement of computer system 600 during the current interval. In some embodiments, interval pace indicator 640c includes a current rate of movement of computer system 600 detected and/or determined by computer system 600 based on information received from one or more sensors of computer system 600. In some embodiments, interval pace indicator 640c includes an average rate of movement of computer system 600 that is determined by computer system 600 based on the detected and/or determined distance traveled during the current interval and/or the amount of time elapsed during the current interval.

At FIG. 6R, workout information region 640 does not include heart rate indicator 640e. In some embodiments, computer system 600 displays heart rate indicator 640e in workout information region 640. In some embodiments, computer system 600 displays heart rate indicator 640e in workout information region 640 in response to one or more user inputs directed to workout user interface 638, such as a swipe gesture, a crown rotation, and/or an air gesture.

At FIG. 6R, computer system 600 detects user input 650o directed to workout user interface 638. In response to detecting user input 650o, computer system 600 displays interval user interface 644, as shown at FIG. 6S.

At FIG. 6S, computer system 600 displays interval user interface 644 while the third interval of the interval portion of the time cycle swimming workout is ongoing. Interval user interface 644 includes time indicator 644a indicating a current time of day and water temperature indicator 644b indicating a temperature of water in which computer system 600 is at least partially submerged. At FIG. 6S, interval user interface 644 include total duration indicator 644c indicating a total amount of time that has elapsed for the time cycle swimming workout (e.g., an amount of time for any completed portions and ongoing portions of the time cycle swimming workout). Interval user interface 644 also includes current interval region 646 and next interval region 648.

Current interval region 646 of interval user interface 644 includes information related to the current interval of the interval portion of the time cycle swimming workout. For instance, current interval region 646 includes distance remaining indicator 646a and interval goal indicator 646b. Distance remaining indicator 646a indicates that there are 52 meters of distance left to be traveled, traversed, and/or moved within the current interval. Interval goal indicator 646b indicates the distance goal (e.g., 100 meters) and the time goal (e.g., 1 minute and 40 seconds) of the current interval of the interval portion of the time cycle swimming workout (e.g., the third interval). Accordingly, the user of computer system 600 can quickly view a summary of the current interval being tracked by computer system 600.

Next interval region 648 of interval user interface 644 includes interval indicator 648a and interval goal indicator 648b. Interval indicator 648a includes a textual indication that the next interval of the time cycle swimming workout includes a work interval. Interval goal indicator 648b indicates the distance goal and the time goal of the next interval. For instance, interval goal indicator 648b indicates that the next interval includes a distance goal of 100 meters and a time goal of one minute and 40 seconds. As such, the user of computer system 600 can quickly view what the next interval will be and/or the goals of the next interval via the interval user interface 644.

At FIG. 6S, computer system 600 detects user input 650p corresponding to interval user interface 644. In response to detecting user input 650p, computer system 600 displays (e.g., re-displays) workout user interface 638.

After computer system 600 displays workout user interface 638 in response to detecting user input 650p, computer system 600 displays workout user interface 638, as shown at FIG. 6T. At FIG. 6T, computer system 600 displays workout user interface 638 at a time that is after a time when computer system 600 displayed workout user interface 638 at FIG. 6R. For instance, overall duration indicator 640a indicates that 11 minutes and 20 seconds have elapsed since computer system 600 initiated the time cycle swimming workout (e.g., as compared to 10 minutes and 40 seconds having elapsed when computer system 600 displays workout user interface 638 at FIG. 6R).

At FIG. 6T, workout information region 640 includes interval duration indicator 640b indicating that 20 seconds remain in the third interval of the interval portion of the time cycle swimming workout. Workout information region 640 includes distance goal duration indicator 640f indicating an amount of time between a start of the current interval (e.g., the third interval) and a current time. At FIG. 6T, distance goal duration indicator 640f indicates that computer system 600 has been tracking and/or detecting a distance of movement of computer system 600 for one minute and 20 seconds. Interval distance indicator 640d indicates that computer system 600 has traveled, traversed, and/or moved a distance of 96 meters during the current interval (e.g., the third interval). In other words, interval distance indicator 640d indicates that computer system 600 has traveled, traversed, and/or moved a distance of 96 meters over one minute and 20 seconds (e.g., the amount of time elapsed during the current interval). At FIG. 6T, interval pace indicator 640c indicates a pace at which computer system 600 has moved over a duration of the current interval.

At FIG. 6T, computer system 600 displays water temperature indicator 638b with an updated value of 62° Fahrenheit (e.g., as compared to 60° Fahrenheit shown at FIGS. 6Q-6S). In some embodiments, computer system 600 receives information about the temperature of an environment in which computer system 600 is positioned via one or more sensors of computer system 600. Based on a determination that computer system 600 initiated a swimming workout, computer system 600 displays water temperature indicator 638b on workout user interface 638. As set forth above, in some embodiments, water temperature indicator 638b includes a real-time indication of a value of the temperature of water in which computer system 600 is at least partially submerged. In some embodiments, computer system 600 receives information related to the temperature of water (e.g., from one or more sensors of computer system 600) at predefined periods of time (e.g., every second, every two seconds, every five seconds, every 10 seconds, every 30 seconds, or every minute). Water temperature indicator 638b therefore provides the user of computer system 600 with an indication of the temperature of the water in which user is swimming.

At FIG. 6U, computer system 600 displays workout user interface 638 at a time that is after a time when computer system 600 displayed workout user interface 638 at FIG. 6T. For instance, overall duration indicator 640a indicates that 11 minutes and 24 seconds have elapsed since computer system 600 initiated the time cycle swimming workout (e.g., as compared to 11 minutes and 20 seconds having elapsed when computer system 600 displays workout user interface 638 at FIG. 6T).

At FIG. 6U, workout information region 640 includes interval duration indicator 640b indicating that 17 seconds remain in the third interval of the interval portion of the time cycle swimming workout. Workout information region 640 includes distance goal duration indicator 640f indicating an amount of time between a start of the current interval (e.g., the third interval) and a current time. At FIG. 6U, distance goal duration indicator 640f indicates that computer system 600 has been tracking and/or detecting a distance of movement of computer system 600 for one minute and 23 seconds. At FIG. 6U, interval pace indicator 640c indicates a pace at which computer system 600 has moved over a duration of the current interval.

Interval distance indicator 640d indicates that computer system 600 has traveled, traversed, and/or moved a distance of 100 meters during the current interval (e.g., the third interval). In other words, interval distance indicator 640d indicates that computer system 600 has traveled, traversed, and/or moved a distance of 100 meters over one minute and 23 seconds (e.g., the amount of time elapsed during the current interval). As set forth above, the distance goal of the third interval of the interval portion of the time cycle swimming workout is 100 meters. As shown at FIG. 6U, when computer system 600 detects and/or determines that the distance goal has been reached, computer system 600 displays completion indicator 640g in workout information region 640 of workout user interface 638. Therefore, the user of computer system 600 can quickly view workout user interface 638 and determine that the distance goal for the current interval has been achieved.

As shown at FIG. 6U, workout information region 640 indicates that 17 seconds remain in the current interval, but that the distance goal of the current interval has been achieved. As set forth above, the time goal of the third interval of the interval portion of the time cycle swimming workout is one minute and 40 seconds. As indicated by distance goal duration indicator 640f, the distance goal was reached and/or achieved within a time of one minute and 23 seconds, which is less than one minute and 40 seconds. Because computer system 600 detects and/or determines that the distance goal of the current interval was reached in a time that is less than the time goal of the current interval, computer system 600 maintains display of workout user interface 638 and does not (e.g., forgoes) initiate the next interval of the interval portion of the time cycle swimming workout. In some embodiments, computer system 600 designates the amount of time remaining in the current interval after the distance goal has been reached as rest time. For instance, the user of computer system 600 was determined to reach a distance of 100 meters within 1 minute and 23 seconds. As such, the user of computer system 600 can rest for 17 seconds before computer system 600 initiates the next interval.

At FIG. 6V, computer system 600 maintains display of workout user interface 638 and continues counting down an amount of time remaining in the current interval. For instance, at FIG. 6V, workout information region 640 includes interval duration indicator 640b indicating that two seconds remain in the third interval of the interval portion of the time cycle swimming workout. Workout information region 640 includes distance goal duration indicator 640f indicating an amount of time between a start of the current interval (e.g., the third interval) and a time at which the distance goal was achieved. At FIG. 6V, distance goal duration indicator 640f indicates that computer system 600 tracked and/or detected that the distance goal was reached in one minute and 23 seconds. At FIG. 6V, computer system 600 stops counting up a time of distance goal duration indicator 640f when the distance goal is reached. For instance, while one minute and 38 seconds have elapsed in the current interval, computer system 600 displays distance goal duration indicator 640f indicating a time of one minute and 23 seconds, which is the time at which the distance goal was reached. At FIG. 6V, interval pace indicator 640c indicates a pace at which computer system 600 moved up until reaching and/or achieving the distance goal. At FIG. 6V, computer system 600 maintains display of completion indicator 640g indicating that the distance goal was reached.

When computer system 600 determines that the amount of time corresponding to the time goal of the third interval elapses, computer system 600 displays interval completion user interface 652, as shown at FIG. 6W. In some embodiments, when computer system 600 determines that the amount of time corresponding to the third interval elapses, computer system 600 initiates the next interval (e.g., the fourth interval) of the interval portion of the time cycle swimming workout and displays workout user interface 638, as shown at FIG. 6X (e.g., without displaying interval completion user interface 652).

At FIG. 6W, interval completion user interface 652 includes interval summary indication 652a and next interval indication 652b. Interval summary indication 652a includes information about the previous interval and/or the interval that was just completed. For instance, at FIG. 6W, interval summary indication 652a includes a time at which computer system 600 detected and/or determined that the distance goal of the last interval was reached (e.g., one minute and 23 seconds). Next interval indication 652b indicates that the next interval to be initiated by computer system includes a fourth interval out of eight intervals. At FIG. 6W, next interval indication 652b includes a distance goal and a time goal associated with the next and/or upcoming interval. As set forth above, the fourth interval of the interval portion of the time cycle swimming workout includes a distance goal of 100 meters and a time goal of one minute and 40 seconds.

At FIG. 6X, computer system 600 displays workout user interface 638 after initiating the fourth interval. At FIG. 6X, computer system 600 has not detected and/or determined that the distance goal of 100 meters has been reached, as indicated by interval distance indicator 640d. For instance, interval distance indicator 640d indicates that computer system 600 has traveled, traversed, and/or moved a distance of 96 meters during the current interval (e.g., the fourth interval). At FIG. 6X, computer system 600 has not detected and/or determined that the distance goal has been reached, but the amount of time corresponding to the time goal of the fourth interval has elapsed. For instance, distance goal duration indicator 640f indicates that computer system 600 has been tracking and/or detecting a distance of movement of computer system 600 for one minute and 42 seconds, which is greater than one minute and 40 seconds. As such, computer system displays interval duration indicator 640b indicating that 2 second have elapsed since the end of the duration corresponding to the time goal (e.g., 2 seconds have elapsed since the one minute and 40 second time goal ended).

At FIG. 6X, computer system 600 displays plus indicator 640h to indicate that an amount of time corresponding to interval duration indicator 640b is beyond a duration of time of the time goal. For instance, at FIG. 6X, plus indicator 640h includes a “+” symbol to indicate that a duration of time of the time goal has expired and that a duration of the current interval exceeds the time goal. In some embodiments, when computer system 600 determines that a duration of the time goal has elapsed before computer system 600 detects that the distance goal is reached, computer system 600 causes interval duration indicator 640b to count up and displays plus indicator 640h.

For instance, at FIG. 6Y, computer system 600 displays workout user interface 638 at a time that is after computer system 600 displays workout user interface 638 at FIG. 6X. At FIG. 6Y, interval duration indicator 640b indicates that 5 seconds have elapsed since a duration of the time goal expired. Computer system 600 displays plus indicator 640h to indicate that the distance goal of the current interval was not reached within a duration of the time goal of the current interval.

At FIG. 6Y, computer system 600 detects that the distance goal of the current interval is reached. For instance, at FIG. 6Y, interval distance indicator 640d indicates that computer system 600 has traveled, traversed, and/or moved a distance of 100 meters during the current interval (e.g., the fourth interval). In some embodiments, computer system 600 displays completion indicator 640g in response to detecting that the distance goal of the current interval is reached. In some embodiments, computer system 600 initiates the next interval of the interval portion of the time cycle swimming workout in response to detecting that the distance goal of the current interval is reached and based on a determination that an amount of time of the current interval is greater than a duration of the time goal of the current interval. In some embodiments, computer system 600 forgoes displaying interval completion user interface 652 in response to detecting that the distance goal of the current interval is reached and based on a determination that an amount of time of the current interval is greater than a duration of the time goal of the current interval.

Turning now to FIG. 6Z, computer system 600 displays workout summary user interface 654 based on a determination that the workout session for the time cycle swimming workout has ended. In some embodiments, computer system 600 determines that the workout session has ended after each portion of the time cycle swimming workout has been completed (e.g., one or more goals of each portion of the time cycle swimming workout have been reached). In some embodiments, computer system 600 determines that the workout session has ended in response to user input requesting to end the workout session. In some embodiments, computer system 600 determines that the workout session has ended in response to an amount of time elapsing since the workout session was first initiated.

At FIG. 6Z, workout summary user interface 654 includes duration indicator 654a, average water temperature indicator 654b and weather information 654c. In some embodiments, workout summary user interface 654 includes additional indications about physical activity information detected and/or determined during the workout session for the time cycle swimming workout. For instance, as set forth below with reference to FIGS. 6AA and 6AB, in some embodiments, computer system 600 displays time information, distance information, pace information, rest information, heart rate information, location information, and/or splits information on workout summary user interface 654.

At FIG. 6Z, duration indicator 654a indicates that computer system 600 initiated the workout session for the time cycle swimming workout at 6:17 am and ended the workout session for the time cycle swimming workout at 7:15 am. Average water temperature indicator 654b indicates that an average temperature of water in which computer system 600 was at least partially submerged was 61° Fahrenheit. In some embodiments, average water temperature indicator 654b includes an average temperature of water detected by one or more sensors of computer system 600 over the duration of the workout session. As set forth above, in some embodiments, computer system 600 detects and/or determines real-time water temperatures via the one or more sensors of computer system. In some embodiments, computer system 600 receives information about the water temperature at predefined periods of time over the course of the workout session. As such, computer system 600 averages two or more water temperature measurements received over the course of the workout session and displays an average water temperature on workout summary user interface 654. At FIG. 6Z, weather information 654c includes an air temperature, such as an average air temperature over the duration of the workout session, a humidity measurement, and/or an air quality measurement corresponding to an environment and/or location in which the workout session was determined to performed (e.g., based on GPS data and/or location information).

In some embodiments, computer system 600 is in communication with an external device, such as device 656. In some embodiments, computer system 600 transmits, sends, and/or communicates information about the workout session to device 656. In some embodiments, device 656 includes display 658, which is larger than display 602 of computer system, such that device 656 can display more information at one time and/or information at a larger size when compared to computer system 600.

At FIG. 6AA, device 656 displays, via display 658, workout summary user interface 660. Workout summary user interface 660 includes interval region 662 and splits region 664. Interval region 662 includes information 662a-662h about respective intervals of the time cycle swimming workout detected and/or tracked by computer system 600. For instance, interval region 662 includes information related to a distance traveled, traversed, and/or moved within a respective interval, a duration in which it took to reach the distance within a respective interval, and/or an amount of rest for a respective interval. As set forth above, in some embodiments, computer system 600 and/or device 656 determines an amount of rest for a respective interval based on an amount of time remaining in a duration of the time goal when the distance goal is determined and/or detected to be reached. For instance, information 662c corresponds to information about the third interval described above with reference to FIGS. 6R-6V. Information 662c indicates that there was 17 seconds of rest in the third interval because computer system 600 detected that the distance goal of 100 meters was reached with 17 seconds remaining in the one minute and 40 seconds time goal. Similarly, information 662d corresponds to information about the fourth interval described above with reference to FIGS. 6X and 6Y. Information 662d indicates that there were 0 seconds of rest in the fourth interval because computer system 600 detected that the distance goal of 100 meters was reached after the one minute and 40 seconds time goal elapsed.

At FIG. 6AA, splits region 664 includes pace information related to different portions of the time cycle swimming workout. In some embodiments, computer system 600 displays pace information for distance increments of the time cycle swimming workout. At FIG. 6AA, computer system displays pace information for different 100 meter distance increments performed during the time cycle swimming workout. In some embodiments, splits region 664 includes information related to a rate of movement over time based on various portions of the time cycle swimming workout for which computer system 600 detected user input requesting a split to occur.

At FIG. 6AA, device 656 detects user input 650q corresponding to workout summary user interface 660. In response to detecting user input 650q, computer system 600 displays heart rate region 666 and location region 668 of workout summary user interface 660, as shown at FIG. 6AB.

At FIG. 6AB, heart rate region 666 includes graphical indication 666a of heart rate measurements and/or information detected, determined, and/or received by computer system 600 over the duration of the workout session of the time cycle swimming workout. Heart rate region 666 also includes heart rate average indicator 666b corresponding to an average heart rate of the user of computer system 600 during the course of the workout session of the time cycle swimming workout. Location region 668 of workout summary user interface 660 includes map indicator 668a, average water temperature indicator 668b, air temperature indicator 668c, humidity indicator 668d, and air quality indicator 668e. In some embodiments, device 656 and/or computer system 600 receives location information over the duration of the workout session and displays map indicator 668a corresponding to the received location information. As set forth above, computer system 600 and device 656 are configured to communicate information between one another, such that device 656 displays average water temperature indicator 668b, air temperature indicator 668c, humidity indicator 668d, and air quality indicator 668e corresponding to average water temperature indicator 654b and weather information 654c in location region 668 of workout summary user interface 660.

FIG. 7 is a flow diagram illustrating a method for updating one or more goals of workout sessions using a computer system in accordance with some embodiments. Method 700 is performed at a computer system (e.g., 100, 300, 500, 600, and/or 656) (e.g., a smart phone, a smart watch, a tablet computer, a laptop computer, a desktop computer, a wearable device, and/or head-mounted device) that is in communication with (e.g., includes and/or is connected to) a display generation component (e.g., 602 and/or 658) (e.g., a display, touch-screen display, a monitor, a holographic display system, and/or a head-mounted display system) and one or more input devices (e.g., 602, 610, and/or 658) (e.g., a touch-sensitive surface (e.g., a touch-sensitive display); a mouse; a keyboard; a remote control; a visual input device (e.g., one or more cameras such as, e.g., an infrared camera, a depth camera, a visible light camera, and/or a gaze tracking camera); an audio input device (e.g., a microphone); a biometric sensor (e.g., a fingerprint sensor, a face identification sensor, a gaze tracking sensor, and/or an iris identification sensor); and/or one or more mechanical input devices (e.g., a depressible input mechanism; a button; a rotatable input mechanism; a crown; and/or a dial)). Some operations in method 700 are, optionally, combined, the orders of some operations are, optionally, changed, and some operations are, optionally, omitted.

As described below, method 700 provides an intuitive way for updating one or more goals of workout sessions. The method reduces the cognitive burden on a user for updating one or more goals of workout sessions, thereby creating a more efficient human-machine interface. For battery-operated computing devices, enabling a user to update one or more goals of workout sessions faster and more efficiently conserves power and increases the time between battery charges.

Prior to initiating a workout session (e.g., workout session of the time cycle swimming workout shown at FIGS. 6Q-6Y) (e.g., a workout tracking function of the computer system that detects and/or determines, via the one or more input devices of the computer system, information related to an active workout being performed by the computer system, such as heart rate, distance, time, calories burned by the user, temperature of an environment in which the workout is being performed, number of steps, and/or number of strokes), the computer system (e.g., 100, 300, 500, 600, and/or 656) prompts (702) (e.g., via distance user interface 620) (e.g., the computer system displays a user interface that includes text, objects, and/or graphical elements providing guidance to a user and/or requesting that the user provide user input) a user of the computer system (e.g., 100, 300, 500, 600, and/or 656) to input (e.g., provide user input that is detected via the one or more input devices of the computer system) a distance (e.g., a length, a measurement, and/or a dimension) associated with the workout session (e.g., a distance of a physical environment in which the user intends and/or will perform a workout for the computer system to track, such as a pool, a track, a lap, a circuit, a field, and/or an ice rink). In some embodiments, the distance has a specific unit of measurement, such as feet, yards, and/or meter. In some embodiments, while the workout session is active and/or while the computer system is performing the workout tracking function, the computer system displays one or more user interface objects corresponding to information associated with the active workout being performed by the user. In some embodiments, the workout session is a swimming workout that tracks a distance in which the user of the computer system swims, an amount of time in which the swimming workout has been performed, a number of calories burned by the user while performing the swimming workout, and/or a distance in which the user of the computer system has swam within a subset of time of the total time in which the swimming workout has been performed.

After (e.g., while) prompting the user of the computer system to input the distance associated with the workout session, the computer system detects (704), via the one or more input devices (e.g., 602, 610, and/or 658), one or more user inputs (e.g., user inputs directed to distance selection user interface object 620b, unit selection user interface object 620c, and/or start user interface object 620d) (e.g., a touch input, an air gesture, a voice command, and/or a button press) selecting (e.g., inputting, designating, and/or choosing) the distance associated with the workout session.

After (e.g., in response to) receiving the one or more user inputs (e.g., user inputs directed to distance selection user interface object 620b, unit selection user interface object 620c, and/or start user interface object 620d) selecting the distance associated with the workout session (706) and in accordance with a determination that the distance associated with the workout session satisfies a set of criteria (e.g., the distance includes a unit of measurement that is consistent with a unit of measurement associated with one or more goals of the workout session, the distance is divisible by a distance goal of the workout session, and/or the distance is consistent with one or more goals of the workout session), the computer system (e.g., 100, 300, 500, 600, and/or 656) initiates (708) the workout session (e.g., activating a workout tracking function of the computer system) (e.g., without displaying the notification with the first selectable user interface object). In some embodiments, the computer system initiates the workout session in accordance with the determination that the distance associated with the workout session satisfies the set of criteria without displaying and/or forgoing displaying the notification.

After (e.g., in response to) receiving the one or more user inputs (e.g., user inputs directed to distance selection user interface object 620b, unit selection user interface object 620c, and/or start user interface object 620d) selecting the distance associated with the workout session (706) and in accordance with a determination that the distance associated with the workout session does not satisfy the set of criteria (e.g., the distance includes a unit of measurement that is not consistent with a unit of measurement associated with one or more goals of the workout session, the distance is not divisible by a distance goal of the workout session, and/or the distance is not consistent with one or more goals of the workout session), the computer system (e.g., 100, 300, 500, 600, and/or 656) displays (710), via the display generation component (e.g., 602 and/or 658), a notification (e.g., 624 and/or 630) (e.g., a user interface and/or one or more user interface objects indicating that the distance associated with the workout session does not satisfy the set of criteria, such as the distance includes a unit of measurement that is different from a unit of measurement of one or more goals of the workout session, the distance is not divisible by a distance goal of the workout session, and/or the distance is not consistent with one or more goals of the workout session) without initiating the workout session, wherein the notification (e.g., 624 and/or 630) includes: a first selectable user interface object (e.g., 624b and/or 630b) (e.g., a first button, selectable icon, affordance, and/or user-interactive user interface element) that, when selected via user input (e.g., 650k and/or 650m), initiates a process to update one or more goals of the workout session (e.g., computer system (e.g., 100, 300, 500, 600, and/or 656) displays goal user interface 626 and/or goal user interface 632) (e.g., automatically updates the one or more goals of the workout session and/or causes the computer system to display (e.g., via the display generation component) a user interface for updating one or more goals of the workout session (e.g., a user interface that includes one or more suggested and/or recommended goals of the workout session that are based on the distance associated with the workout session and/or based on one or more initial and/or original goals of the workout session)). Initiating the workout session in accordance with the determination that the distance associated with the workout session satisfies the set of criteria and displaying the notification in accordance with the determination that the distance associated with the workout session does not satisfy the set of criteria allows the computer system to start the workout session and/or notify a user of the computer system that the one or more goals of the workout session can be updated, thereby providing improved visual feedback to the user and/or performing an operation when a set of conditions has been met without requiring further user input.

In some embodiments, while displaying the first selectable user interface object (e.g., 624b and/or 630b) (e.g., in accordance with the determination that the distance associated with the workout session does not satisfy the set of criteria), the computer system (e.g., 100, 300, 500, 600, and/or 656) detects, via the one or more input devices (e.g., 602, 610, and/or 658), user input (e.g., 650k and/or 650m) (e.g., a touch input, an air gesture, a voice command, and/or a button press) corresponding to selection (e.g., inputting, designating, and/or choosing) of the first selectable user interface object (e.g., 624b and/or 630b). In response to detecting the user input (e.g., 650k and/or 650m) corresponding to selection of the first selectable user interface object (e.g., 624b and/or 630b), the computer system (e.g., 100, 300, 500, 600, and/or 656) displays, via the display generation component (e.g., 602 and/or 658), a goal user interface (e.g., 626 and/or 632) (e.g., a user interface that includes one or more indicators corresponding to respective goals of the workout session, a user interface that enables a user of the computer system to change and/or update one or more goals of the workout session, a user interface that includes suggestions for one or more goals of the workout session, a user interface that includes suggestions for updating one or more goals of the workout session based on the distance associated with the workout session, and/or a user interface that provides information about changes made to one or more goals of the workout session based on the distance associated with the workout session), wherein the goal user interface (e.g., 626 and/or 632) includes: an indication of an initial goal of the workout session (e.g., 628a, 634a, and/or 636a) (e.g., a goal of the workout session that was input, selected, and/or determined prior to receiving the one or more user inputs selecting the distance associated with the workout session) and an indication of an updated goal of the workout session (e.g., 628b, 634b, and/or 636b) (e.g., a goal of the workout session, such as a distance, a time, and/or a pace, in which the computer system updates, modifies, and/or suggests based on the distance associated with the workout session), different from the initial goal of the workout session, wherein the updated goal of the workout session is based on the distance associated with the workout session (e.g., the updated goal of the workout session is updated to a different unit of measurement, the updated goal of the workout session is updated to a different distance that is divisible by the distance associated with the workout session, the updated goal of the workout session is updated to a different time to account for a change in a unit of measurement and/or a distance of a distance goal that is based on the distance associated with the workout session, and/or the updated goal of the workout session is updated to a different rate to account for a change in a unit of measurement and/or a distance of a distance goal that is based on the distance associated with the workout session). Displaying the goal user interface that includes an indication of an initial goal of the workout session and an indication of an updated goal of the workout session provides a suggested goal to the user of the computer system based on the distance associated with the workout session, thereby providing improved visual feedback to the user.

In some embodiments, the goal user interface (e.g., 626 and/or 632) further includes a selectable start user interface object (e.g., 626d and/or 632d) (e.g., a button, selectable icon, affordance, and/or user-interactive user interface element) that, when selected via user input (e.g., 650n) (e.g., touch input, air gesture, and/or mouse pointer), initiates (e.g., using the updated goal and, optionally, without using the initial goal) the workout session (e.g., causes, such as automatically causes, the computer system to begin tracking activity information and/or data associated with the user performing a workout). In some embodiments, the process to activate the workout session automatically activates the workout session without requiring further user inputs. The goal user interface including a selectable start user interface object enables a user of the computer system to quickly initiate the workout session without requiring the user to navigate to another user interface, thereby reducing the number of inputs needed to perform an operation.

In some embodiments, the indication of the initial goal of the workout session (e.g., 628a, 634a, and/or 636a) includes a first appearance (e.g., a strikethrough text effect as shown at FIGS. 6L and 6O) (e.g., a first emphasis, text effect, size, color, and/or font), and the indication of the updated goal of the workout session (e.g., 628b, 634b, and/or 636b) includes a second appearance (e.g., no strikethrough text effect as shown at FIGS. 6L and 6O) (e.g., a second emphasis, text effect, size, color, and/or font) that is different from the first appearance. In some embodiments, the indication of the initial goal of the workout session includes a strikethrough text effect and the indication of the update goal of the workout session does not include a strikethrough text effect. The indication of the initial goal of the workout session including a first appearance and the indication of the updated goal of the workout session including a second appearance that is different from the first appearance enables a user of the computer system to distinguish between the initial goal and the updated goal, thereby providing improved visual feedback to the user.

In some embodiments, the initial goal is a distance goal of an interval of the workout session (e.g., a 100 meter distance goal as shown at FIG. 6O) (e.g., a distance of a predetermined portion of the workout session, a sub-distance of a total distance of the workout session, and/or a distance that corresponds to a duration of the workout session that is less than a total duration of the workout session). In some embodiments, the updated goal is an updated distance goal of the interval of the workout session. In some embodiments, the computer system adjusts the initial goal of the interval of the workout session to the updated goal of the interval of the workout session after detecting the user input corresponding to selection of the first selectable user interface object. The initial goal being a distance goal of an interval of the workout session enables a user of the computer system to customize the workout session and allows the computer system to provide updated goals for a portion of the workout session based on the distance associated with the workout session, thereby providing improved customization of workout sessions and providing improved visual feedback to the user.

In some embodiments, the initial goal is a distance goal of a total distance of the workout session (e.g., a distance corresponding to a total and/or entire duration of the workout session, a distance in which the user intends to travel and/or traverse before ending the workout session, and/or a distance that, when the computer system detects has been reached, causes the computer system to end the workout session and/or output a notification to the user of the computer system). In some embodiments, the updated goal is an updated distance goal of the total distance of the workout session. In some embodiments, the computer system adjusts the initial goal of the workout session to the updated goal of the workout session after detecting the user input corresponding to selection of the first selectable user interface object. The initial goal being a distance goal of a total distance of the workout session enables a user of the computer system to customize the workout session and allows the computer system to provide updated goals for a total distance of the workout session based on the distance associated with the workout session, thereby providing improved customization of workout sessions and providing improved visual feedback to the user.

In some embodiments, while displaying the first selectable user interface object (e.g., 624b and/or 630b) (e.g., in accordance with the determination that the distance associated with the workout session does not satisfy the set of criteria), the computer system (e.g., 100, 300, 500, 600, and/or 656) detects, via the one or more input devices (e.g., 602, 610, and/or 658), user input (e.g., 650k and/or 650m) (e.g., a touch input, an air gesture, a voice command, and/or a button press) corresponding to selection (e.g., inputting, designating, and/or choosing) of the first selectable user interface object (e.g., 624b and/or 630b). In response to detecting the user input (e.g., 650k and/or 650m) corresponding to selection of the first selectable user interface object (e.g., 624b and/or 630b), the computer system (e.g., 100, 300, 500, 600, and/or 656) updates (e.g., without requiring further user inputs) the one or more goals of the workout session (e.g., automatically adjusting one or more initial goals of the workout session to one or more updated goals of the workout session based on the distance associated with the workout session). In some embodiments, the computer system updates the one or more goals of the workout session by adjusting a unit of measurement of the one or more goals of the workout session, a distance of the one or more goals of the workout session, a duration for completing the one or more goals of the workout session, and/or a pace for completing the one or more goals of the workout session. In some embodiments, the computer system initiates a process for activating the workout session in response to detecting the user input corresponding to selection of the first selectable user interface object. Updating the one or more goals of the workout session in response to detecting user input corresponding to selection of the first selectable user interface object allows the computer system to quickly change and/or modify the one or more goals of the workout session based on the distance associated with the workout session without requiring additional user input, thereby reducing the number of inputs needed to perform an operation.

In some embodiments, the notification (e.g., 624 and/or 630) further includes a second selectable user interface object (e.g., 624c and/or 630c) (e.g., a second button, selectable icon, affordance, and/or user-interactive user interface element) that, when selected via user input (e.g., 650j), ceases display of the notification (e.g., 624 and/or 630) (e.g., automatically stops and/or discontinues display of the notification) without updating the one or more goals of the workout session. In some embodiments, ceasing display of the notification includes the computer system displaying a different user interface, such as a user interface associated with configuring the workout session. In some embodiments, ceasing display of the notification includes the computer system initiating the workout session without updating the one or more goals of the workout session. The notification including a second selectable user interface object that is configured to cease display of the notification without updating the one or more goals of the workout session enables a user to determine whether to update the one or more goals of the workout session without requiring the user to navigate to another user interface, thereby reducing the number of inputs needed to perform an operation.

In some embodiments, initiating the workout session includes the computer system (e.g., 100, 300, 500, 600, and/or 656) displaying, via the display generation component (e.g., 602 and/or 658), a countdown indicator (e.g., 622, 622a, and/or 622b) (e.g., text, an image, an object, and/or a graphical element that changes in appearance over a predetermined period of time before the computer system starts tracking activity information corresponding to the workout session) that counts down for a period of time (e.g., 1 second, 2 seconds, 3 seconds, or 5 seconds). In some embodiments, the computer system displays the countdown indicator for the period of time and then ceases display of the countdown indicator. In some embodiments, after the period of time, the computer system transitions from displaying the countdown indicator to displaying a workout user interface. Displaying a countdown indicator that counts down for a period of time enables a user to prepare for the start of the workout session without requiring the user to provide additional input, thereby performing an operation when a set of conditions has been met without requiring further user input.

In some embodiments, after initiating the workout session, the computer system (e.g., 100, 300, 500, 600, and/or 656) displays, via the display generation component (e.g., 602 and/or 658), a workout user interface (e.g., 638) (e.g., a user interface that includes one or more user interface objects indicating an amount of time that has elapsed since the workout session was started, a detected distance in which the user of the computer system has traveled and/or traversed since the start of the workout session, and/or activity information detected by the computer system since the start of the workout session, such as a heart rate, distance, time, calories burned by the user, temperature of an environment in which the workout is being performed, number of steps, and/or number of strokes) that includes information (e.g., 640a-640h) corresponding to physical activity performed by a user of the computer system (e.g., 100, 300, 500, 600, and/or 656) (e.g., an amount of time spent performing a workout of the workout session, a distance traveled and/or traversed, heart rate, time, calories burned by the user, temperature (e.g., detected via one or more sensors of the computer system) of an environment (e.g., water temperature and/or air temperature) in which the workout is being performed, number of steps, and/or number of strokes when swimming). Displaying a workout user interface after initiating the workout session enables a user to view a summary of information detected, tracked, and/or determined by the computer system during the workout session without requiring the user to navigate to another user interface, thereby reducing the number of inputs needed to perform an operation and/or performing an operation when a set of conditions has been met without requiring further user input.

In some embodiments, the workout user interface (e.g., 638) includes an indication of a temperature of water (e.g., 638b) (e.g., the workout session corresponds to a swimming workout in which the computer system (and, optionally, the user of the computer system) is at least partially submerged within water and the computer system detects (e.g., via one or more sensors of the computer system) and/or receives information related to a temperature, such as a current temperature and/or an average temperature over time, of the water in which the computer system is at least partially submerged). The workout user interface including an indication of a temperature of water provides a user of the computer system with information about a temperature of water during the workout session, thereby providing improved visual feedback to the user.

In some embodiments, the computer system (e.g., 100, 300, 500, 600, and/or 656) detects, via the one or more input devices (e.g., 602, 610, and/or 658), the temperature of the water (e.g., the computer system is configured to be in communication with a sensor that measures, detects, estimates, and/or determines a temperature of an environment in which the computer system and/or a user of the computer system is located, such as water when the user of the computer system is swimming). Detecting the temperature of the water via the one or more input devices that are in communication with the computer system allows the computer system to provide information about the temperature of the water to the user without requiring an additional device, thereby providing improved visual feedback to the user and/or reducing the number of devices required to perform an operation.

In some embodiments, while displaying the workout user interface (e.g., 638) and in accordance with a determination that a set of criteria is met (e.g., user input 650o) (e.g., the computer system detects one or more user inputs, the workout session includes one or more intervals and/or portions, having respective distance goals and/or duration goals, and/or a goal of the workout session has been met), the computer system (e.g., 100, 300, 500, 600, and/or 656) ceases display of the workout user interface (e.g., 638) (e.g., stopping and/or discontinuing displaying the workout user interface and/or a portion of the workout user interface) and the computer system (e.g., 100, 300, 500, 600, and/or 656) displays, via the display generation component (e.g., 602 and/or 658), an interval user interface (e.g., 644 and/or 652) (e.g., a user interface that includes information about one or more intervals and/or portions of the workout session that include respective distance goals and/or duration goals, such as information about a current interval and/or portion of the workout session, information about a previous and/or completed interval and/or portion of the workout session, and/or information about an upcoming and/or next interval and/or portion of the workout session). In some embodiments, in accordance with a determination that the set of criteria is not met, the computer system maintains display of the workout user interface without displaying the interval user interface. In some embodiments, in accordance with a determination that the set of criteria is not met, the computer system ceases display of the workout user interface and displays another user interface that includes information about the workout session. Ceasing display of the workout user interface and displaying the interval user interface in accordance with a determination that set of criteria is met allows the computer system to display information that is relevant to the user without requiring the user to provide additional input, thereby performing an operation when a set of conditions has been met without requiring further user input and/or providing improved visual feedback to the user.

In some embodiments, the interval user interface (e.g., 644 and/or 652) includes an indication (e.g., 646a) (e.g., text, an object, an image, and/or a graphical element) of a remaining goal amount (e.g., a remaining amount of distance and/or a remaining amount of time) of a first goal (e.g., a distance goal and/or a duration goal) of a current interval of the workout session (e.g., a portion of the workout session that does not include the entire workout session, such as a portion of a total distance of the workout session and/or a portion of a total duration of the workout session, that is currently being tracked by the computer system and/or performed by a user of the computer system) and an indication (e.g., 648b) (e.g., text, an object, an image, and/or a graphical element) of a second goal (e.g., a distance goal and/or a duration goal that is the same as the first goal or different from the first goal) of a next interval of the workout session (e.g., a portion of the workout session that does not include the entire workout session, such as a portion of a total distance of the workout session and/or a portion of a total duration of the workout session, that the computer system is not currently tracking, that the computer system has not activated and/or initiated, and/or that the user of the computer system is not yet performing). The interval user interface including an indication of a remaining goal amount of a first goal of a current interval of the workout session and an indication of a second goal of a next interval of the workout session allows the computer system to display information that is relevant to the user without requiring the user to provide additional input, thereby performing an operation when a set of conditions has been met without requiring further user input and/or providing improved visual feedback to the user.

In some embodiments, the workout user interface (e.g., 638) includes an indication of a temperature of water (e.g., 638b) (e.g., the workout session corresponds to a swimming workout in which the user of the computer system is at least partially submerged within water and the computer system receives information related to a temperature, such as a current temperature and/or an average temperature, of the water in which the user of the computer system is at least partially submerged) and the interval user interface (e.g., 644 and/or 652) includes the indication of the temperature of water (e.g., 644b) (e.g., display of the indication of the temperature of water is maintained on the interval workout user interface even when the workout user interface ceases to be displayed). The workout user interface and the interval user interface including the indication of the temperature of water enables the user of the computer system to determine the temperature of the water without requiring the user to navigate to another user interface and/or provide additional user input, thereby reducing the number of inputs needed to perform an operation and/or providing improved visual feedback to the user.

In some embodiments, after initiating the workout session and in accordance with a determination that a set of end criteria has been met (e.g., the computer system detects one or more user inputs requesting to end the workout session, a duration of the workout session has elapsed, a distance of the workout session has been reached, and/or all intervals of the workout session have been completed), the computer system (e.g., 100, 300, 500, 600, and/or 656) initiates a process to end the workout session (e.g., as described with reference to FIG. 6Y) (e.g., automatically ceasing to track, detect, and/or record activity information corresponding to physical activity of the user of the computer system that occurs during the workout session) and the computer system (e.g., 100, 300, 500, 600, and/or 656) displays, via the display generation component (e.g., 602 and/or 658), a workout summary user interface (e.g., 654 and/or 660) (e.g., a user interface that includes information recorded, tracked, detected, and/or about the workout session, such as a duration of the workout session, a distance traveled and/or traversed during the workout session, activity information tracked and/or detected during the workout session, information corresponding to durations and/or distances traveled and/or traversed during intervals of the workout session, average activity information over the course of a total duration of the workout session, and/or information about an environment in which the computer system was located during the workout session). Initiating a process to end the workout session and displaying a workout summary user interface after initiating the workout session and in accordance with a determination that a set of end criteria has been met allows the computer system to end and/or cease the workout session and display information that is relevant to the user without requiring the user to provide additional input and/or navigate to another user interface, thereby performing an operation when a set of conditions has been met without requiring further user input and/or providing improved visual feedback to the user.

In some embodiments, the workout summary user interface (e.g., 654 and/or 660) includes an indication of an average water temperature (e.g., 654b and/or 668b) (e.g., detected via the one or more input devices of the computer system) over a duration (e.g., the total duration or less than the total duration) of the workout session (e.g., an average water temperature detected via the one or more input devices of the computer system over a total duration of the workout session). In some embodiments, the workout session includes a swimming workout in which the computer system and/or a user of the computer system are at least partially submerged in water, the one or more input devices of the computer system detect, measure, estimate, and/or determine a temperature of the water throughout a duration of the workout session, and/or the computer system averages two or more temperatures of the water that were detected, measured, estimated, and/or determined over the course of the total duration of the workout session. The workout summary user interface including an indication of an average water temperature over a duration of the workout session allows the computer system to display information that is relevant to the user without requiring the user to provide additional input, thereby providing improved visual feedback to the user.

In some embodiments, the workout session is a swimming workout session (e.g., the computer system tracks, detects, estimates, measures, and/or determines activity information associated with a user of the computer system as the user swims in a pool, a pond, a lake, a river, and/or an ocean). The workout session being a swimming workout session allows the computer system to provide customizable workout sessions and/or more accurately track a physical activity being performed by the user, thereby improving the customization of the computer system and/or providing improved visual feedback to the user.

Note that details of the processes described above with respect to method 700 (e.g., FIG. 7) are also applicable in an analogous manner to the methods described below. For example, method 800 optionally includes one or more of the characteristics of the various methods described above with reference to method 700. For example, the one or more goals updated by the computer system in method 700 can be used in the intervals of the active workout session of method 800. For brevity, these details are not repeated below.

FIG. 8 is a flow diagram illustrating a method for displaying information about respective intervals of workout sessions using a computer system in accordance with some embodiments. Method 800 is performed at a computer system (e.g., 100, 300, 500, 600, and/or 656) (e.g., a smart phone, a smart watch, a tablet computer, a laptop computer, a desktop computer, a wearable device, and/or head-mounted device) that is in communication with (e.g., includes and/or is connected to) a display generation component (e.g., 602 and/or 658) (e.g., a display, touch-screen display, a monitor, a holographic display system, and/or a head-mounted display system) and one or more input devices (e.g., 602, 610, and/or 658) (e.g., a touch-sensitive surface (e.g., a touch-sensitive display); a mouse; a keyboard; a remote control; a visual input device (e.g., one or more cameras such as, e.g., an infrared camera, a depth camera, a visible light camera, and/or a gaze tracking camera); an audio input device (e.g., a microphone); a biometric sensor (e.g., a fingerprint sensor, a face identification sensor, a gaze tracking sensor, and/or an iris identification sensor); and/or one or more mechanical input devices (e.g., a depressible input mechanism; a button; a rotatable input mechanism; a crown; and/or a dial)). Some operations in method 800 are, optionally, combined, the orders of some operations are, optionally, changed, and some operations are, optionally, omitted.

As described below, method 800 provides an intuitive way for displaying information about respective intervals of workout sessions. The method reduces the cognitive burden on a user for viewing information about respective intervals of workout sessions, thereby creating a more efficient human-machine interface. For battery-operated computing devices, enabling a user to view information about respective intervals of workout sessions faster and more efficiently conserves power and increases the time between battery charges.

While detecting, via the one or more input devices (e.g., 602, 610, and/or 658), activity information (e.g., information indicated by workout information 640a-640h) (e.g., heart rate, distance, time, calories burned by the user, temperature of an environment in which the workout is being performed, number of steps, and/or number of strokes) associated with an active workout session (e.g., the workout session for the time cycle swimming workout shown at FIGS. 6Q-6Y) (e.g., the computer system is performing a workout tracking function, where the computer system displays one or more user interface objects corresponding to the activity information associated with an active workout being performed by the user) that includes a first interval (e.g., the interval described with reference to FIGS. 6R-6W) (e.g., a first subset of the active workout session that does not include the entirety of a predetermined program and/or goal of the active workout session, such as a subset of a total distance goal of the active workout session and/or a subset of a total time goal of the active workout session) corresponding to a first distance goal (e.g., a first swimming distance goal, running distance goal, walking distance goal, cycling distance goal, rowing distance goal, and/or hiking distance goal) and a first duration (e.g., a first range of time beginning at the start and/or initiation of the active workout session and/or a first range of time beginning at the start and/or initiation of the first interval, such as a portion and/or subset, of the active workout session) and a second interval (e.g., the interval described with reference to FIGS. 6X-6Y) (e.g., a second subset of the active workout session that does not include the entirety of a predetermined program and/or goal of the active workout session, such as a subset of a total distance goal of the active workout session and/or a subset of a total time goal of the active workout session) corresponding to a second distance goal (e.g., a second swimming distance goal, running distance goal, walking distance goal, cycling distance goal, rowing distance goal, and/or hiking distance goal) and a second duration (e.g., a second range of time beginning at the start and/or initiation of the active workout session and/or a second range of time beginning at the start and/or initiation of the first interval, such as a portion and/or subset, of the active workout session), the computer system (e.g., 100, 300, 500, 600, and/or 656) receives (800), via the one or more input devices (e.g., 602, 610, and/or 658), an indication that the first distance goal corresponding to the first interval of the active workout session has been reached (e.g., as described with reference to FIGS. 6U and 6Y) (e.g., the computer system receives information, via the one or more input devices, indicating that the user has reached the first distance goal). In some embodiments, the active workout session is a swimming workout, where the computer system tracks a distance in which the user of the computer system swims, an amount of time in which the swimming workout has been performed, a number of calories burned by the user while performing the swimming workout, and/or a distance in which the user of the computer system has swam within a subset of time of the total time in which the swimming workout has been performed. In some embodiments, the first distance goal includes a distance goal for a portion of the active workout session, such as a subset of the active workout session. In some embodiments, the first distance goal is set and/or selected by the user of the computer system and/or the first distance goal is a default distance for the active workout session.

In response to receiving the indication that the first distance goal corresponding to the first interval of the active workout session has been reached (804) and in accordance with a determination that the first distance goal corresponding to the first interval of the active workout session has been reached within the first duration (e.g., as described with reference to FIGS. 6U-6W) (e.g., the computer system determines that the user has traveled and/or traversed the first distance goal within a range of time beginning at the start and/or initiation of the active workout session and/or a range of time beginning at the start and/or initiation of the first interval, such as a portion and/or subset, of the active workout session) corresponding to the first interval of the active workout session (e.g., one minute and 40 seconds, as described with reference to FIGS. 6R-6Y), the computer system (e.g., 100, 300, 500, 600, and/or 656) displays (806), via the display generation component (e.g., 602 and/or 658), an indication (e.g., 640b) of an amount of time remaining (e.g., a numeric indication and/or a graphical non-numeric indication) in the first duration corresponding to the first interval of the active workout session (e.g., an amount of time from a current time until a time at which the duration of time ends and/or is scheduled to end) without initiating the second interval of the active workout session (e.g., the interval described with reference to FIGS. 6X-6Y) (e.g., without initiating the second duration and/or tracking the second distance of the second interval and/or without beginning to track and/or detect activity information associated with a second subset of the active workout session and/or without alerting and/or notifying the user of the computer system that the second interval of the active workout session has started), wherein the computer system (e.g., 100, 300, 500, 600, and/or 656) is configured to cease updating (e.g., cease displaying and/or cease decrementing/incrementing) the indication (e.g., 640b) of the amount of time remaining in the first duration after the first duration ends (e.g., the indication of the amount of time remaining in the first duration corresponding to the first interval of the active workout session counts down the remaining time in the first duration until the first duration ends and/or the computer system ceases display of the indication of the amount of time remaining in the first duration corresponding to the first interval of the active workout session in response to the first duration ending and/or lapsing).

In response to receiving the indication that the first distance goal corresponding to the first interval of the active workout session has been reached (804) and in accordance with a determination that the first distance goal corresponding to the first interval of the active workout session has not been reached within the first duration corresponding to the first interval of the active workout session (e.g., as described with reference to FIGS. 6X-6Y) (e.g., the computer system determines that the user has taken longer than the first duration amount of time to travel and/or traverse the first distance goal), the computer system (e.g., 100, 300, 500, 600, and/or 656) initiates (808) the second interval of the active workout session (e.g., the interval described with reference to FIGS. 6X-6Y and/or a next interval after the interval described with reference to FIGS. 6X-6Y) (e.g., the computer system initiates the second duration corresponding to the second interval of the workout session and/or begins tracking the second distance corresponding to the second interval of the workout session once the computer system detects that the user has reached the first distance goal because the first duration has elapsed). In some embodiments, initiating the second interval of the workout session includes the computer system tracking and/or detecting activity information associated with a subset and/or portion of the active workout session and/or the computer system notifying and/or alerting the user of the computer system that the second interval of the workout session has started. In some embodiments, before the first distance goal is reached and after the first duration has elapsed, the computer system displays, via the display generation component, an amount of time that has passed since the first duration lapsed before initiating the second interval of the active workout session. In some embodiments, in accordance with a determination that the first distance goal corresponding to the first interval of the active workout session has been reached within the first duration and in accordance with a determination that the first duration has elapsed, the computer system initiates the second interval of the active workout session.

Displaying the indication of the amount of time remaining in the first duration corresponding to the first interval of the active workout session without initiating the second interval of the active workout session in accordance with a determination that the first distance goal corresponding to the first interval of the active workout session has been reached within the first duration and initiating the second interval of the active workout session in accordance with a determination that the first distance goal corresponding to the first interval of the active workout session has not been reached within the first duration, enables a user of the computer system to determine whether the first distance goal corresponding to the first interval was reached within the first duration corresponding to the first interval, to determine whether the user has additional time within the first duration after reaching the first distance goal corresponding to the first interval, and/or whether to start the second interval of the active workout session, thereby providing improved visual feedback to the user and/or performing an operation when a set of conditions has been met without requiring further user input.

In some embodiments, while displaying the indication (e.g., 640b) of the amount of time remaining in the first duration, the computer system (e.g., 100, 300, 500, 600, and/or 656) detects an end of the first duration (e.g., an amount of time that corresponds to the first duration elapsed and/or passed since a start of the first duration and/or the computer system detects that the amount of time corresponding to the first duration has ended). In response to detecting the end of the first duration, the computer system initiates a timer (e.g., a timer associated with interval duration indicator 640b) (e.g., a function of the computer system that tracks and/or records an amount of time that has passed from a predetermined time) corresponding to the second duration of the second interval (e.g., the computer system initiates the second interval by beginning to track and/or cause the second duration corresponding to the second interval to elapse). In some embodiments, the computer system displays a user interface object indicating an amount of time that has passed since the second duration of the second interval started and/or a user interface object indicating an amount of time remaining in the second duration of the second interval. Initiating a timer corresponding to the second duration of the second interval in response to detecting the end of the first duration allows the computer system to begin tracking and/or detecting information about the second interval without requiring additional user input, thereby reducing the number of inputs needed to perform an operation and/or performing an operation when a set of conditions has been met without requiring further user input.

In some embodiments, prior to detecting the activity information associated with the active workout session (e.g., before the computer system initiates the active workout session and/or begins tracking and/or detecting the activity information of the active workout session), the computer system (e.g., 100, 300, 500, 600, and/or 656) detects, via the one or more input devices (e.g., 602, 610, and/or 658), user input (e.g., 650d) (e.g., a touch input, an air gesture, a voice command, and/or a button press) corresponding to a request to adjust a characteristic of a respective interval corresponding to the active workout session (e.g., the computer system receives one or more user inputs adjusting a respective distance goal, a respective duration, a respective type of interval, and/or a respective swimming stroke corresponding to the interval). In response to detecting the user input (e.g., 650d) corresponding to the request to adjust the characteristic of the respective interval corresponding to the active workout session, the computer system (e.g., 100, 300, 500, 600, and/or 656) adjusts the characteristic of the respective interval corresponding to the active workout session. Adjusting the characteristic of the respective interval corresponding to the active workout session in response to detecting the user input corresponding to the request to adjust the characteristic of the respective interval corresponding to the active workout session enables a user of the computer system to customize the active workout session based on a fitness level of the user, thereby improving the customization of the computer system.

In some embodiments, the characteristic of the respective interval corresponding to the active workout session includes a respective distance goal (e.g., a number, amount, and/or unit of measurement, such as meters or yards, of the first distance goal and/or the second distance goal). The characteristic of the respective interval corresponding to the active workout session being a respective distance goal enables a user of the computer system to customize the active workout session based on a fitness level of the user, thereby improving the customization of the computer system.

In some embodiments, the characteristic of the respective interval corresponding to the active workout session includes a respective duration (e.g., a number, amount, and/or unit of measurement, such as seconds, minutes, or hours, of the first duration and/or the second duration). The characteristic of the respective interval corresponding to the active workout session being a respective duration enables a user of the computer system to customize the active workout session based on a fitness level of the user, thereby improving the customization of the computer system.

In some embodiments, the characteristic of the respective interval corresponding to the active workout session includes a swimming stroke type (e.g., a type of swimming stroke, such as freestyle, backstroke, butterfly, kick, breast stroke, breast kick, sidestroke, and/or elementary backstroke, in which the computer system is configured to detect and/or track during the respective duration of the respective interval of the active workout session). The characteristic of the respective interval corresponding to the active workout session being a swimming stroke type enables a user of the computer system to customize the active workout session based on a fitness level of the user, thereby improving the customization of the computer system.

In some embodiments, the characteristic of the respective interval corresponding to the active workout session includes one or more goals of a warmup or a cooldown of the active workout session (e.g., a distance goal and/or a duration of a warmup interval or a cooldown interval of the active workout session). The characteristic of the respective interval corresponding to the active workout session being a one or more goals of a warmup or cooldown of the active workout session enables a user of the computer system to customize the active workout session based on a fitness level of the user, thereby improving the customization of the computer system.

In some embodiments, while displaying the indication (e.g., 640b) of the amount of time remaining in the first duration (e.g., in accordance with a determination that the first distance goal corresponding to the first interval of the active workout session has been reached before the first duration has not ended and/or in accordance with a determination that the first duration has not ended), the computer system (e.g., 100, 300, 500, 600, and/or 656) detects, via the one or more input devices (e.g., 602, 610, and/or 658), user input (e.g., 650o) (e.g., a touch input, an air gesture, a voice command, and/or a button press) requesting to display an interval user interface (e.g., 644) (e.g., a user interface that includes information about one or more intervals and/or portions of the workout session that include respective distance goals and/or duration goals, such as information about a current interval and/or portion of the workout session, information about a previous and/or completed interval and/or portion of the workout session, and/or information about an upcoming and/or next interval and/or portion of the workout session). In response to detecting the user input (e.g., 650o) requesting to display the interval user interface (e.g., 644) (and, optionally, in accordance with a determination that the first duration has not ended), the computer system (e.g., 100, 300, 500, 600, and/or 656) displays the interval user interface (e.g., 644). In some embodiments, displaying the interval user interface includes the computer system transitioning from displaying a workout user interface (e.g., a user interface that includes one or more user interface objects indicating an amount of time that has elapsed since the workout session was started, a detected distance in which the user of the computer system has traveled and/or traversed since the start of the workout session, and/or activity information detected by the computer system since the start of the workout session, such as a heart rate, distance, time, calories burned by the user, temperature of an environment in which the workout is being performed, number of steps, and/or number of strokes) to displaying the interval user interface. In some embodiments, displaying the interval user interface includes the computer system ceasing display of the workout user interface and displaying the interval user interface. Displaying the interval user interface in response to detecting the user input requesting to display the interval user interface enables a user of the computer system to determine a status of a current interval of the active workout session and/or one or more goals of a next interval of the active workout session, thereby providing improved visual feedback to the user.

In some embodiments, displaying the interval user interface (e.g., 644) includes the computer system (e.g., 100, 300, 500, 600, and/or 656) displaying (e.g., concurrently and/or sequentially), an indication (e.g., 646a) (e.g., text, images, objects, and/or graphical elements) of an amount of progress (e.g., a distance traveled within the first duration corresponding to the first interval, an amount of time that has elapsed within the first duration corresponding to the first interval, a remaining amount of distance, and/or a remaining amount of time) toward reaching one or more first goals (e.g., the first distance goal and/or the first duration) corresponding to the first interval and an indication (e.g., 648a and/or 648b) (e.g., text, images, objects, and/or graphical elements) of one or more second goals (e.g., the second distance goal, the second duration, a type of interval of the second interval, and/or a swimming stroke type corresponding to the second interval) corresponding to the second interval. The interval user interface including an indication of an amount of progress toward reaching one or more first goals corresponding to the first interval and an indication of one or more second goals corresponding to the second interval enables a user of the computer system to determine a status of a current interval of the active workout session and/or one or more goals of a next interval of the active workout session, thereby providing improved visual feedback to the user.

In some embodiments, in response to receiving the indication that the first distance goal corresponding to the first interval of the active workout session has been reached and in accordance with the determination that the first distance goal corresponding to the first interval of the active workout session has been reached within the first duration corresponding to the first interval of the active workout session, the computer system (e.g., 100, 300, 500, 600, and/or 656) displays, via the display generation component (e.g., 602 and/or 658), an indication (e.g., 640g) (e.g., text, an image, an object, and/or a graphical element, such as a check mark) that the first distance goal has been reached (e.g., has been reached within the first duration corresponding to the first interval of the active workout session). In some embodiments, in accordance with the determination that the first distance goal corresponding to the first interval of the active workout session has not been reached within the first duration corresponding to the first interval of the active workout session, the computer system forgoes display of the indication that the first distance goal has been reached. Displaying the indication that the first distance goal has been reached in accordance with a determination that the first distance goal corresponding to the first interval of the active workout session has been reached within the first duration corresponding to the first interval of the active workout session enables a user of the computer system to quickly and easily determine that the user reached the first distance goal within the first duration, thereby providing improved visual feedback to the user.

In some embodiments, the computer system (e.g., 100, 300, 500, 600, and/or 656) detects an end of the first duration corresponding to the first interval (e.g., an amount of time that corresponds to the first duration elapsed and/or passed since a start of the first duration and/or the computer system detects that the amount of time corresponding to the first duration has ended). In response to detecting the end of the first duration corresponding to the first interval of the active workout session prior to receiving the indication that the first distance goal corresponding to the first interval of the active workout session has been reached (e.g., the first distance goal corresponding to the first interval of the active workout session was not reached within the first duration corresponding to the first interval and/or the user of the computer system took a longer amount of time to traverse and/or travel the first distance goal than the amount of time corresponding to the first duration), the computer system (e.g., 100, 300, 500, 600, and/or 656) displays, via the display generation component (e.g., 602 and/or 658), an indication (e.g., 640b and/or 640h) (e.g., text, an image, an object, and/or a graphical element) of an amount of time that has passed since the first duration ended (e.g., an amount of second, minutes, and/or hours that have passed since the first duration ended without the computer system detecting that the first distance goal corresponding to the first interval of the workout session having been reached), wherein the computer system (e.g., 100, 300, 500, 600, and/or 656) is configured to cease updating (e.g., cease displaying and/or cease decrementing/incrementing) the indication (e.g., 640b and/or 640h) of the amount of time that has passed since the first duration ended when the computer system receives an indication that the first distance goal corresponding to the first interval of the active workout session has been reached (e.g., the indication of the amount of time that has passed since the first duration ended counts up once the first duration ends). Displaying an amount of time that has passed since the first duration ended in response to detecting the end of the first duration corresponding to the first interval prior to receiving the indication that the first distance goal corresponding to the first interval has been reached enables a user of the computer system to quickly and easily determine that the first distance goal has not been reached within the first duration, thereby providing improved visual feedback to the user.

In some embodiments, in response to detecting that a set of end criteria has been met for the active workout session (e.g., the computer system detects one or more user inputs requesting to end the workout session, a duration of the workout session has elapsed, a distance of the workout session has been reached, and/or all intervals of the workout session have been completed), the computer system (e.g., 100, 300, 500, 600, and/or 656) initiates a process to end the active workout session (e.g., as described with reference to FIGS. 6Y and 6Z) (e.g., automatically ceasing to track, detect, and/or record activity information corresponding to physical activity of the user of the computer system that occurs during the workout session) and the computer system (e.g., 100, 300, 500, 600, and/or 656) displays, via the display generation component (e.g., 602 and/or 658), a workout summary user interface (e.g., 654 and/or 660) (e.g., a user interface that includes information recorded, tracked, detected, and/or about the workout session, such as a duration of the workout session, a distance traveled and/or traversed during the workout session, activity information tracked and/or detected during the workout session, information corresponding to durations and/or distances traveled and/or traversed during intervals of the workout session, average activity information over the course of a total duration of the workout session, and/or information about an environment in which the computer system was located during the workout session). Initiating a process to end the active workout session and displaying a workout summary user interface in response to detecting that a set of end criteria has been met for the active workout session allows the computer system to end and/or cease the workout session and display information that is relevant to the user without requiring the user to provide additional input and/or navigate to another user interface, thereby performing an operation when a set of conditions has been met without requiring further user input and/or providing improved visual feedback to the user.

In some embodiments, displaying the workout summary user interface (e.g., 654 and/or 660) includes the computer system (e.g., 100, 300, 500, 600, and/or 656) displaying an indication (e.g., 662a-662h) (e.g., text, one or more objects, one or more images, and/or one or more graphical elements) of a comparison (e.g., a difference) between an amount of time taken to reach a respective distance goal corresponding to a respective interval and a respective duration corresponding to the respective interval (e.g., a comparison of an amount of time in which the computer system detected that it took to reach the a distance goal to the duration of the interval, such as an amount of time that the user was able to rest between completing the distance goal and the end of the duration of the interval and/or an amount of time in which it took the user to complete the distance goal beyond the duration of the interval). The workout summary user interface including an indication of a comparison between an amount of time take to reach a respective distance goal corresponding to a respective interval and a respective duration corresponding to the respective interval enables a user of the computer system to quickly and easily determine whether the user reached a goal within a respective amount of time for different portions of the active workout session, thereby providing improved visual feedback to the user.

In some embodiments, displaying the workout summary user interface (e.g., 654 and/or 660) includes the computer system (e.g., 100, 300, 500, 600, and/or 656) displaying an indication (e.g., text, one or more objects, one or more images, and/or one or more graphical elements) of a pace (e.g., 662a-662h and/or 664) (e.g., a rate of travel and/or movement over time) corresponding to the first interval and an indication of a pace corresponding to the second interval. In some embodiments, the workout summary user interface includes indications of respective paces for each interval (e.g., the first interval, the second interval, and/or additional intervals) of the active workout session (e.g., the computer system displays a pace in which the computer system detected and/or determined for the respective distance goal to be reached for each interval of the active workout session). In some embodiments, the computer system further displays an average pace corresponding to the detected total distance traveled and/or traversed over a total amount of time corresponding to the active workout session. The workout summary user interface including an indication of a pace corresponding to the first interval and an indication of a pace corresponding to the second interval enables a user of the computer system to quickly and easily view information about different portions of the active workout session, thereby providing improved visual feedback to the user.

In some embodiments, the active workout session is a swimming workout session (e.g., the computer system tracks, detects, estimates, measures, and/or determines activity information associated with a user of the computer system as the user swims in a pool, a pond, a lake, a river, and/or an ocean). The workout session being a swimming workout session allows the computer system to provide customizable workout sessions and/or more accurately track a physical activity being performed by the user, thereby improving the customization of the computer system and/or providing improved visual feedback to the user.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve workout sessions. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide more accurate information related to a workout session. Accordingly, use of such personal information data enables users to view more relevant information about a workout session. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of physical activity information and/or workout information, the present technology can be configured to allow users to select to “opt in” or “opt out” of participation in the collection of personal information data during registration for services or anytime thereafter. In another example, users can select not to provide and/or share physical activity information and/or workout information. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, physical activity information and/or workout session information can be generalized based on non-personal information data or a bare minimum amount of personal information, such as the content being requested by the device associated with a user, other non-personal information, or publicly available information.

What is claimed is:

1. A computer system configured to communicate with a display generation component and one or more input devices, comprising:
one or more processors; and
memory storing one or more programs configured to be executed by the one or more processors, the one or more programs including instructions for:
prior to initiating a workout session, prompting a user of the computer system to input a distance associated with the workout session;
after prompting the user of the computer system to input the distance associated with the workout session, detecting, via the one or more input devices, one or more user inputs selecting the distance associated with the workout session; and
after receiving the one or more user inputs selecting the distance associated with the workout session:
in accordance with a determination that the distance associated with the workout session satisfies a set of criteria, initiating the workout session; and
in accordance with a determination that the distance associated with the workout session does not satisfy the set of criteria, displaying, via the display generation component, a notification without initiating the workout session, wherein the notification includes:
a first selectable user interface object that, when selected via user input, initiates a process to update one or more goals of the workout session.

2. The computer system of claim 1, wherein the one or more programs further include instructions for:
while displaying the first selectable user interface object, detecting, via the one or more input devices, user input corresponding to selection of the first selectable user interface object; and
in response to detecting the user input corresponding to selection of the first selectable user interface object, displaying, via the display generation component, a goal user interface, wherein the goal user interface includes:
an indication of an initial goal of the workout session; and
an indication of an updated goal of the workout session, different from the initial goal of the workout session, wherein the updated goal of the workout session is based on the distance associated with the workout session.

3. The computer system of claim 2, wherein the goal user interface further includes a selectable start user interface object that, when selected via user input, initiates the workout session.

4. The computer system of claim 2, wherein:
the indication of the initial goal of the workout session includes a first appearance, and
the indication of the updated goal of the workout session includes a second appearance that is different from the first appearance.

5. The computer system of claim 2, wherein the initial goal is a distance goal of an interval of the workout session.

6. The computer system of claim 2, wherein the initial goal is a distance goal of a total distance of the workout session.

7. The computer system of claim 1, wherein the one or more programs further include instructions for:
while displaying the first selectable user interface object, detecting, via the one or more input devices, user input corresponding to selection of the first selectable user interface object; and
in response to detecting the user input corresponding to selection of the first selectable user interface object, updating the one or more goals of the workout session.

8. The computer system of claim 1, wherein the notification further includes a second selectable user interface object that, when selected via user input, ceases display of the notification without updating the one or more goals of the workout session.

9. The computer system of claim 1, wherein initiating the workout session includes:
displaying, via the display generation component, a countdown indicator that counts down for a period of time.

10. The computer system of claim 1, wherein the one or more programs further include instructions for:
after initiating the workout session, displaying, via the display generation component, a workout user interface that includes information corresponding to physical activity performed by a user of the computer system.

11. The computer system of claim 10, wherein the workout user interface includes an indication of a temperature of water.

12. The computer system of claim 11, wherein the one or more programs further include instructions for:
detecting, via the one or more input devices, the temperature of the water.

13. The computer system of claim 10, wherein the one or more programs further include instructions for:
while displaying the workout user interface:
in accordance with a determination that a set of criteria is met:
ceasing display of the workout user interface; and
displaying, via the display generation component, an interval user interface.

14. The computer system of claim 13, wherein the interval user interface includes:
an indication of a remaining goal amount of a first goal of a current interval of the workout session; and
an indication of a second goal of a next interval of the workout session.

15. The computer system of claim 13, wherein:
the workout user interface includes an indication of a temperature of water, and
the interval user interface includes the indication of the temperature of water.

16. The computer system of claim 1, wherein the one or more programs further include instructions for:
after initiating the workout session and in accordance with a determination that a set of end criteria has been met:
initiating a process to end the workout session; and
displaying, via the display generation component, a workout summary user interface.

17. The computer system of claim 16, wherein the workout summary user interface includes an indication of an average water temperature over a duration of the workout session.

18. The computer system of claim 1, wherein the workout session is a swimming workout session.

19. A non-transitory computer-readable storage medium storing one or more programs configured to be executed by one or more processors of a computer system that is in communication with a display generation component and one or more input devices, the one or more programs including instructions for:
prior to initiating a workout session, prompting a user of the computer system to input a distance associated with the workout session;
after prompting the user of the computer system to input the distance associated with the workout session, detecting, via the one or more input devices, one or more user inputs selecting the distance associated with the workout session; and
after receiving the one or more user inputs selecting the distance associated with the workout session:
in accordance with a determination that the distance associated with the workout session satisfies a set of criteria, initiating the workout session; and
in accordance with a determination that the distance associated with the workout session does not satisfy the set of criteria, displaying, via the display generation component, a notification without initiating the workout session, wherein the notification includes:
a first selectable user interface object that, when selected via user input, initiates a process to update one or more goals of the workout session.

20. A method, comprising:
at a computer system that is in communication with a display generation component and one or more input devices:
prior to initiating a workout session, prompting a user of the computer system to input a distance associated with the workout session;
after prompting the user of the computer system to input the distance associated with the workout session, detecting, via the one or more input devices, one or more user inputs selecting the distance associated with the workout session; and
after receiving the one or more user inputs selecting the distance associated with the workout session:
in accordance with a determination that the distance associated with the workout session satisfies a set of criteria, initiating the workout session; and
in accordance with a determination that the distance associated with the workout session does not satisfy the set of criteria, displaying, via the display generation component, a notification without initiating the workout session, wherein the notification includes:
a first selectable user interface object that, when selected via user input, initiates a process to update one or more goals of the workout session.
